(19) United States
(12) Patent Application Publication
(10) Pub. No.: US 2019/0133682 A1
(43) Pub. Date: May 9, 2019

(54) ABLATION CATHETER TIPS AND CATHETERS

(71) Applicants: HADASIT MEDICAL RESEARCH SERVICES AND DEVELOPMENT LTD., Jerusalem (IL); YISSUM RESEARCH DEVELOPMENT COMPANY OF THE HEBREW UNIVERSITY OF JERUSALEM LTD., Jerusalem (IL)

(72) Inventors: Eyal ITSHAYEK, Macabim (IL); Ram ELAZARY, Jerusalem (IL); Eitan MELAMED, Haifa (IL); Noam MEYUHAS, Jerusalem (IL); Amnon BUXBOIM, Tel Aviv (IL); Yoav MINTZ, Jerusalem (IL); Elchanan FRIED, Jeusalem (IL); Gahl LEVY, Ramat Gan (IL)

(21) Appl. No.: 16/304,615

(22) PCT Filed: Jun. 15, 2017
(86) PCT No.: PCT/IL2017/050667

§ 371 Date: Nov. 26, 2018

Related U.S. Application Data
(60) Provisional application No. 62/350,746, filed on Jun. 16, 2016.

(51) Int. Cl. A61B 18/14; A61B 18/16

(52) U.S. Cl. CPC A61B 18/1492; A61B 2018/00577; A61B 2218/007; A61B 18/16

(57) ABSTRACT

Disclosed herein are ablation catheter tips and catheters, and catheter ablation methods, wherein a same channel within the catheter may be simultaneously used both for applying suction and attaching a target tissue to the ablation electrode, and for expelling a coolant introduced into the catheter via a second channel, fluidly coupled thereto.

### TECHNICAL FIELD

The present disclosure generally relates to the field of ablation catheters.

### BACKGROUND

Atrial fibrillation (AF) is a heart rhythm disorder characterized by rapid and chaotic electrical activity in the atria. Atrial electrical signals bombard the atrioventricular (AV) node, with some of the signals propagating therethrough to the ventricles to produce a rapid and irregular heart-rate, often causing symptoms of palpitations, shortness of breath, and/or fatigue. AF may lead to heart failure. AF may also lead to blood stagnation in the atria and, as a result, to the formation of blood clots, which may travel to the brain through the arteries and cause a stroke. AF affects more than 2 million people in the U.S. alone; its incidence increasing with age.

Treatment of AF includes clot formation prevention, slowing the heart-rate, and cardioversion—regulating the heart-rate to restore and maintain normal sinus rhythm. Controlling the heart rate and maintaining sinus rhythm is difficult and often unsuccessful. Preventing clot formation with anti-coagulants carries the risk of major hemorrhage.

Catheter ablation has been used to treat heart rhythm disorders for more than 20 years now, with its use in treating AF increasing in recent years. A thin catheter tube is inserted into a vein, typically in the groin, and guided through the inferior vena cava into the right atrium, wherefrom it may be guided, via the septum, into the left atrium. The catheter tube's tip is placed against a target tissue on the heart wall. A radiofrequency (RF) electrical current is applied through an ablation electrode on the catheter tube's tip, heating the electrode to produce a small burn in the target tissue of about 6 to 8 mm in diameter. In treatment of heart rate arrhythmias, the electrical source of the arrhythmia is ablated. In treatment of AF, the catheter tube's tip is placed near the exit of a pulmonary vein. The ablation is performed repeatedly in order to burn and scar a ring of tissue surrounding the exit of the pulmonary vein. The ablation procedure is then repeated at the exit of another pulmonary vein. The resulting scarred tissue has poor electrical conductance, and hence acts as a barrier, obstructing or eliminating passage therethrough of the chaotic electrical signals causing the AF.

### SUMMARY

Aspects of the disclosure, in some embodiments thereof, relate to cardiac ablation catheters and catheter tips. More specifically, aspects of the disclosure, in some embodiments thereof, relate to endo-cardiac ablation catheters, wherein a same channel may simultaneously serve both for securing the target tissue to the ablation electrode and for expelling a coolant, which is used to prevent excessive heating of the ablation electrode.

Catheter ablation for treating AF is both complex and challenging. A first challenge involves achieving a secure coupling between the ablation electrode and the target tissue, such that any motion of the ablation electrode relative to the target tissue is kept to a minimum. The challenge is made difficult by the movement of cardiac tissue resulting from the contraction and expansion of the heart. A static or near static coupling of the ablation electrode to the target tissue may allow for controllably forming uniform lesions.

A second challenge involves preventing excessive heating of the target tissue and related damage. Two techniques for dealing with excessive heat production include closed loop cooling and open loop cooling. In closed loop cooling, the ablation electrode is cooled by propagating a coolant (a cool fluid) through the catheter. In particular, the coolant does not come into contact with the target tissue. In contrast, in open loop cooling, the coolant is at least partially discharged outside the catheter tip, cooling the ablation electrode, as well as the target tissue. A problem associated with open loop cooling is that of excessive hydration, where up to 3 liters of a crystalloid solution (coolant) may be discharged into the heart, and thereby into the vascular system, during an endo-cardiac ablation operation. Rapidly infusing such an amount of crystalloid solution may dilute the blood and significantly increase the intravascular volume, which is undesirable, especially in older subjects whose hearts typically have a lower tolerance to fluid overload.

The present disclosure describes several ways to achieve a sufficiently static coupling (attachment) between the ablation electrode and the target tissue such as to generate thick and uniform lesions, which additionally incorporate advantages of open and closed loop cooling. Similarly to closed loop cooling, substantially none of the irrigant (e.g. the coolant) is released into bodily cavities (e.g. the atria) Similarly to open loop cooling, tissue surrounding the ablation electrode is irrigated directly. Additional advantages include (i) the removal of ablation byproducts (e.g. char) and/or the prevention/reduction in the rate of formation of ablation byproducts, and (ii) continuous feedback regarding the security of the coupling between the ablation electrode and the target tissue by continuously checking whether the expelled irrigant is blood-free.

Each of the disclosed ablation catheters includes an inlet channel for introducing an irrigant (e.g. a coolant or any other fluid) into the catheter, and an outlet channel (i.e. a vacuum channel), configured to be coupled to a vacuum source, for applying suction via a suction port at the catheter tip. Advantageously, the outlet channel is fluidly coupled to the inlet channel at the catheter tip, thereby serving also to expel the coolant from the catheter.

The catheter tip is configured such that by applying suction when the catheter tip is suitably positioned close to a tissue ablation site: (a) the ablation electrode is secured to the tissue ablation site, and (b) the suction port (as well as any other port on the catheter tip) is covered by adjacent tissue to the tissue ablation site. The covering results in the formation of a closed irrigation zone, that is to say, a space within and about the catheter tip, which is fluidly disconnected by the tissue from bodily cavities, such as the left atrium chamber.

When passing through the closed irrigation zone, the coolant comes into (direct or indirect) thermal contact with the ablation electrode, thereby cooling the ablation electrode and the tissue ablation site. In particular, some of the coolant will wash against the adjacent tissue blocking the suction port, thereby cooling the adjacent tissue and helping to confine the heating to the tissue ablation site. Advantageously, blood in the closed irrigation zone may be washed away by the coolant prior to commencing the ablation. During ablation, blood in the proximity of the ablation electrode and target tissue may lead to the formation of blood clots, as well as to a reduction in the ablation electrode's conductivity as organic material solidifies over the ablation electrode.

Further, the monitoring of the expelled coolant for signs of blood provides continuous feedback regarding the security of the coupling between the ablation electrode and the target tissue. Persistence of blood in the expelled coolant may indicate failure to securely attach the ablation electrode to the target tissue. A sudden appearance of blood in the expelled coolant, following a continuous period during the ablation wherein the expelled coolant was clear, may indicate that the ablation electrode is no longer securely attached to the target tissue.

According to an aspect of some embodiments, there is provided an ablation catheter tip including
A tip body, having a proximal tip body end and a distal tip body end.
An inlet channel, having a proximal inlet channel end and a distal inlet channel end, the inlet channel being longitudinally disposed within the tip body.
An outlet channel, having a proximal outlet channel end and a distal outlet channel end, the outlet channel being longitudinally disposed within the tip body.
A suction port, located at the distal tip body end and fluidly coupled to the distal outlet channel end.
An ablation electrode positioned at the distal tip body end;

The suction port is configured to secure a target tissue, at a tissue ablation site on the target tissue, to the ablation electrode by applying a vacuum force via the outlet channel when the distal tip body end is proximate to or in contact with the target tissue.

The inlet channel and the outlet channel are fluidly coupled at the distal tip body end such that the fluid coupling is maintained when the suction port is covered, thereby facilitating propagating a fluid from the inlet channel to the outlet channel and expelling the fluid via the proximal outlet channel end, when the vacuum force secures (i) the ablation electrode to the tissue ablation site and (ii) tissue, adjacent to the tissue ablation site, to the suction port.

According to some embodiments, the ablation electrode may be moved relative to the distal tip body end such as to facilitate coupling of the ablation electrode to the tissue ablation site, without compromising the vacuum. According to some embodiments, the ablation electrode is moveable relative to the distal tip body. For example, the ablation electrode may be movable within a static tip body (or the distal tip body) and/or the tip body (or the distal tip body) may be movable with respect to a static ablation electrode.

According to some embodiments, the movement may be radially, axially, and/or longitudinally. Longitudinally movement ay include distal movement and/o radial movement. According to some embodiments, the ablation electrode may protrude distally from the distal tip body end by longitudinally moving the ablation electrode in a distal direction. Such movement may be manual or automatic and/or may be facilitated by a steerable/maneuverable element such as a sheath located, for example, between the ablation electrode and the tip body (which may also be referred to as the delivery catheter). Optionally, the ablation electrode may be marked by mark scale to facilitate evaluation of the protrusion range.

According to some embodiments, the distal tip body end is configured to induce direct and/or indirect thermal coupling between the ablation electrode and a fluid present at the distal inlet channel end, at the distal outlet channel end, and/or in between the channels at the distal tip body end, and thereby to controllably effect a temperature of the ablation electrode by propagating the fluid at a controllable introduction temperature via the inlet channel and the outlet channel, through the distal tip body end.

According to some embodiments, the inlet channel and the outlet channel are fluidly connected via an opening, duct, or recess.

According to some embodiments, the fluid is a coolant.

According to some embodiments, the inlet channel extends between the proximal tip body end and the distal tip body end.

According to some embodiments, the outlet channel extends between the proximal tip body end and the distal tip body end.

According to some embodiments, the suction port at least partially circumscribes the ablation electrode.

According to some embodiments, the tip body and the inlet channel are tubular, and the outlet channel is defined by the tip body and inlet channel, and a space between the tip body and the inlet channel.

According to some embodiments, the inlet channel further includes an inlet channel cap, mounted on the distal inlet channel end, and at least one fluid opening, located at the distal inlet channel end. The ablation electrode is positioned in/on the inlet channel cap, such as to be at least partially exposed, and the fluid opening fluidly connects the inlet channel to the outlet channel.

According to some embodiments, the at least one fluid opening includes two or more fluid openings, which are annularly disposed about the inlet channel.

According to some embodiments, the ablation catheter tip further includes an inlet tube longitudinally disposed within the tip body, extending from a proximal inlet tube end to a distal inlet tube end, and an inner core longitudinally disposed within the inlet tube.

The outlet channel is defined by the tip body and the inlet tube, and includes a first space between the tip body and the inlet tube. The inlet channel is defined by the inlet tube and the inner core, and includes a second space between the inlet tube and the inner core. The tip body extends distally farther than the inlet tube. The inner core extends distally at least as much as the tip body. The ablation electrode is positioned on/in a core tip of the inner core.

According to some embodiments, the ablation catheter tip further includes
A second inlet channel, having a proximal second inlet channel end and a distal second inlet channel end, the second inlet channel being longitudinally disposed within the tip body.
A second outlet channel, having a proximal second outlet channel end and a distal second outlet channel end, the second outlet channel being longitudinally disposed within the tip body.
A second suction port, located at the distal tip body end and fluidly coupled to the distal second outlet channel end.

The distal tip body end includes four recesses, each of the recesses extending from a respective proximal inlet channel end to a respective distal outlet channel end, such as to circumscribe the ablation electrode, the recesses being configured to maintain fluid connectivity between the inlet channels and the outlet channels when the recesses, the inlet channel ends, and the suction ports are covered at a distal tip body extremity of the distal tip body.

According to some embodiments, the distal inlet channel ends and the distal outlet channels ends are arranged in a square-like configuration, with each of the inlet channels being adjacent to both of the outlet channels.

According to some embodiments, the ablation catheter tip is configured to be mounted on a distal end of a catheter tubing assembly.

According to some embodiments, the ablation catheter tip may be used in the treatment of AF.

According to an aspect of some embodiments, there is provided an ablation catheter including
An elongate member, being flexible, having a proximal member end and a distal member end.
An inlet channel, having a proximal inlet channel end and a distal inlet channel end, at least a distal portion of the inlet channel being longitudinally disposed within the elongate member.
An outlet channel, having a proximal outlet channel end and a distal outlet channel end, the outlet channel being longitudinally disposed within the elongate member.
A suction port mounted on the distal outlet channel end.
A vacuum port, mounted on the proximal outlet channel end.
A fluid inlet port mounted on the proximal inlet channel end.
An ablation electrode positioned at the distal member end.

The fluid inlet port is configured to be fluidly coupled to a fluid source. The vacuum port is configured to be fluidly coupled to a vacuum source. The suction port is configured to secure a target tissue, at a tissue ablation site on the target tissue, to the ablation electrode by applying a vacuum force via the outlet channel when the distal member end is proximate to or in contact with the target tissue.

The inlet channel and the outlet channel are fluidly coupled at the distal member end such that the fluid coupling is maintained when the suction port is covered, thereby facilitating propagating a fluid from the inlet channel to the outlet channel and expelling the fluid via the vacuum port, when the vacuum force secures (i) the ablation electrode to the tissue ablation site and (ii) a tissue, adjacent to the tissue ablation site, to the suction port.

According to some embodiments, the distal member end is configured to induce direct and/or indirect thermal coupling between the ablation electrode and a fluid present at the distal inlet channel end, at the distal outlet channel end, and/or in between the channels at the distal member end, and thereby to controllably effect a temperature of the ablation electrode by propagating the fluid at a controllable introduction temperature via the inlet channel and the outlet channel, through the distal member end.

According to some embodiments, the ablation catheter may be used in the treatment of AF.

According to an aspect of some embodiments, there is provided a catheter ablation method including the steps of
Inserting into a subject's body a catheter including
   A catheter tip with an ablation electrode positioned on/in a distal end thereof.
   An inlet channel and an outlet channel both extending along the catheter until the catheter tip distal end and fluidly coupled at the catheter tip distal end.
   A suction port mounted on the catheter tip distal end and fluidly coupled to the outlet channel.
   The fluid coupling of the inlet channel and outlet channel at the catheter tip distal end is maintained when the suction port is covered.
Positioning and orienting the catheter tip, such that the ablation electrode faces a tissue ablation site on a target tissue in a body cavity.
Securing the ablation electrode to the tissue ablation site by applying a vacuum force along the outlet channel, thereby covering the suction port with a tissue, adjacent to the tissue ablation site, and fluidly sealing the catheter tip from the body cavity.
Propagating an irrigant through the inlet channel and the outlet channel, via the catheter tip distal end, wherein the irrigant washes against the adjacent tissue covering the suction port.
Ablating the tissue at the tissue ablation site.

According to some embodiments, the irrigant is a coolant and the catheter tip distal end is configured such that heat generated by the ablation electrode is transferred to the coolant when the coolant flows through the catheter tip distal end, thereby effecting a temperature of the ablation electrode in the step of propagating the irrigant.

According to some embodiments, the step of ablating the tissue includes inducing a current through the ablation electrode.

According to some embodiments, the ablation catheter method further includes, prior to the step of ablating, testing for a presence of blood in the coolant expelled via the inlet channel. If the presence of blood persists: significantly decreasing the flow of the coolant, switching off the vacuum force, and repeating the step of positioning and orienting and subsequent steps.

According to some embodiments, the ablation catheter method further includes, following the step of ablating, monitoring a temperature of the ablation electrode. If the temperature exceeds a threshold temperature: switching off the current, increasing the flow of the coolant, and switching on the current again.

According to some embodiments, the ablation catheter method further includes, following the step of ablating, if there remain tissue ablation sites that have not been ablated: repeating the step of positioning and orienting the catheter tip and subsequent steps with respect to another tissue ablation site.

According to some embodiments, the ablation catheter method is for use in treatment of atrial fibrillation.

It will be understood by the skilled person that the embodiments disclosed herein may also be used for other applications beyond endo-cardiac ablation, such as epi-cardiac ablation, as well as for applications beyond cardiac ablation, involving coupling between an operative element or medical probe and a target tissue.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically depicts an ablation catheter inserted into the heart, according to some embodiments;

FIG. 2A schematically depicts an ablation catheter tip, according to some embodiments;

FIG. 2B schematically depicts a cross-sectional view from A-A of the ablation catheter tip of FIG. 2A, with an ablation electrode secured against a target tissue, according to some embodiments;

FIG. 2C schematically depicts the cross-sectional view of FIG. 2B with an irrigant flowing in the ablation catheter tip, according to some embodiments;

FIG. 3A schematically depicts an embodiment of an ablation catheter tip, according to some embodiments;

FIG. 3B schematically depicts a cross-sectional view from A-A of the ablation catheter tip of FIG. 3A, with an ablation electrode secured against a target tissue, according to some embodiments;

FIG. 4A schematically depicts an embodiment of an ablation catheter tip, according to some embodiments;

FIG. 4B schematically depicts a cross-sectional view from A-A of the ablation catheter tip of FIG. 4A, with an ablation electrode secured against a target tissue, according to some embodiments;

FIG. 5A schematically depicts an ablation catheter tip, according to some embodiments;

FIG. 5B schematically depicts a cross-sectional view from B-B of the ablation catheter tip of FIG. 5A, with an ablation electrode secured against a target tissue and with an irrigant flowing in the ablation catheter tip, according to some embodiments;

FIG. 6A schematically depicts a side-view of an ablation catheter tip, according to some embodiments;

FIG. 6B schematically depicts a top-view of the ablation catheter tip of FIG. 6A, according to some embodiments;

FIG. 6C schematically depicts a cross-sectional view from C-C (defined in FIG. 6B) of the ablation catheter tip of FIG. 6A, according to some embodiments;

FIG. 6D schematically depicts a cross-sectional view from D-D (defined in FIG. 6B) of the ablation catheter tip of FIG. 6A, according to some embodiments;

FIG. 6E schematically depicts the cross-sectional view of FIG. 6C, with an ablation electrode secured against a target tissue and with an irrigant flowing in the ablation catheter tip, according to some embodiments;

FIG. 6F schematically depicts the cross-sectional view of FIG. 6D, with the ablation electrode secured against the target tissue and with the irrigant flowing in the ablation catheter tip, according to some embodiments;

FIG. 6G schematically depicts a top view of an embodiment of an ablation catheter tip, according to some embodiments;

FIG. 7A schematically depicts an ablation catheter, according to some embodiments;

FIG. 7B schematically depicts the ablation catheter of FIG. 7A, according to some embodiments;

FIG. 7C schematically depicts a cross-sectional view from E-E defined in FIG. 7B) of the ablation catheter of FIG. 7A, according to some embodiments;

FIG. 7D schematically depicts a cross-sectional view from F-F defined in FIG. 7B) of an ablation catheter tip of the ablation catheter of FIG. 7A, according to some embodiments;

FIG. 7E schematically depicts a cross-sectional view from G-G defined in FIG. 7B) of the catheter tubing assembly of FIG. 7A, according to some embodiments;

FIG. 8 schematically depicts a block diagram describing an ablation setup including an ablation catheter, according to some embodiments;

FIG. 9 schematically depicts a flow chart describing a method for catheter ablation, according to some embodiments.

FIG. 10A schematically depicts a side-view delivery catheter of a catheter ablation assembly, according to some embodiments;

FIG. 10B schematically depicts a cross-sectional front-view from K-K of the delivery catheter of FIG. 10A, according to some embodiments;

FIG. 10C schematically depicts a back-view of the delivery catheter of FIG. 10A, according to some embodiments;

FIG. 10D schematically depicts a cross-sectional side-view from L-L (defined in FIG. 10C) of the delivery catheter of FIG. 10A, according to some embodiments;

FIG. 10E schematically depicts the cross-sectional view of FIG. 10D, with a catheter ablation tube inserted into the delivery catheter, according to some embodiments; and

FIG. 10F schematically depicts the cross-sectional view of FIG. 10E, with an ablation electrode secured against a target tissue, and with an irrigant flowing through the catheter insertion tube and through an outlet channel in the delivery catheter, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

As used herein, according to some embodiments, the term “cooling” with respect to a cooling of a first object/medium by a second object/medium, having a lower temperature than the first object/medium, refers to a transfer of heat from a first object/medium to the second object/medium. The transfer of heat may result in a lowering of the temperature of the first object/medium, or not, as, for example, may happen if the first object/medium is simultaneously being heated.

As used herein, according to some embodiments, the term “drawn to each other” with respect to a first object and a second object may refer to both objects moving towards each other, or to only one of the objects moving towards the other object, which remains at rest.

As used herein, according to some embodiments, the terms “tip body” and “member” are used interchangeably.

As used herein, according to some embodiments, the terms “to cover” and “to block” are used interchangeably.

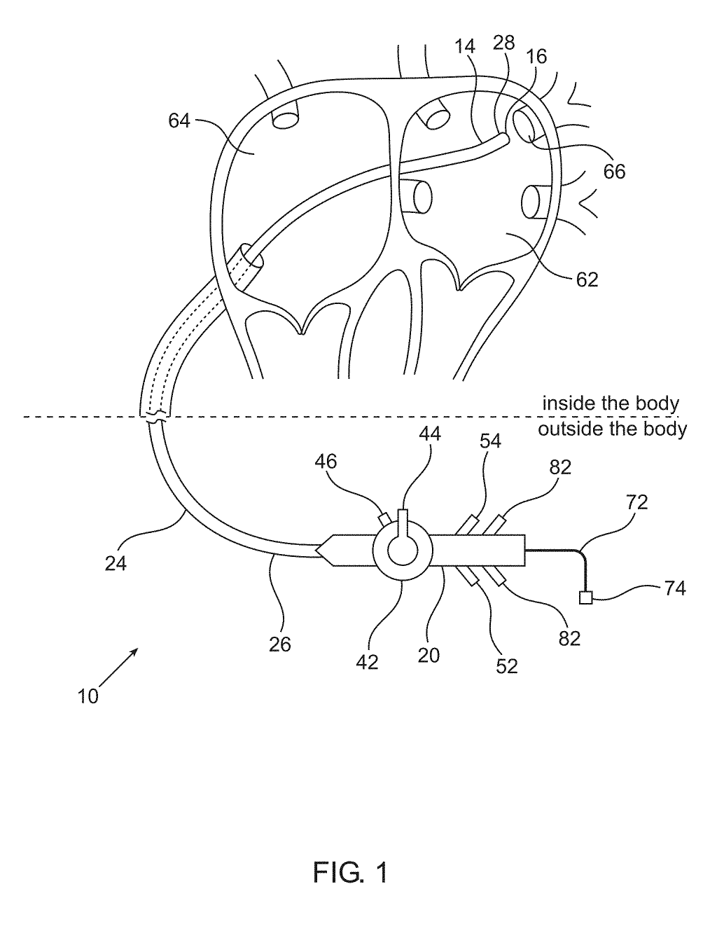

FIG. 1 schematically depicts an embodiment of an ablation catheter 10 including an ablation catheter tip 14, according to some embodiments of the present disclosure. Ablation catheter tip 14 includes an ablation electrode 16. Ablation catheter 10 further includes a catheter handle 20 and an elongate member 24, having a proximal member end 26 and a distal member end 28. Elongate member 24 is flexible and is attached on proximal member end 26 to catheter handle 20. Catheter tip 14 is mounted on distal member end 28.

Elongate member 24 includes an inlet channel and an outlet channel (both not shown), both extending through elongate member 24. Catheter handle 20 includes a steering mechanism 42 for steering ablation catheter tip 14. Steering mechanism 42 includes a steering lever 44 and a locking lever 46. According to some embodiments, steering lever 44 serves to deflect ablation catheter tip 14 on a pre-determined arc (not shown), while locking lever 46 may be used to fix the deflection angle. By, in addition, rotating catheter handle 20, ablation catheter tip 14 may be steered on a pre-determined hemisphere (not shown). Such steering mechanisms are well known in the art and will not be elaborated on herein.

Catheter handle 20 further includes a fluid inlet port 52 for introducing fluids into the inlet channel, such as a coolant (cooling fluid) to cool ablation electrode 16, as elaborated on hereinbelow. In addition, catheter handle 20 includes a vacuum port 54 configured to be coupled to a vacuum source. Vacuum port 54 is fluidly connected to a suction port (not shown) on ablation catheter tip 14, via the outlet channel. The suction port is configured to secure ablation electrode 16 to a target tissue, as elaborated on hereinbelow.

Elongate member 24 is shown inserted into a subject's left atrium 62, via the right atrium 64, such that ablation catheter tip 14 is located at a pulmonary vein opening 66. Ablation catheter tip 14 is positioned such that ablation electrode 16 is secured to a tissue ablation site on a target tissue (both not indicated) located at pulmonary vein opening 66.

An electrical wire 72 extends through catheter handle 20 and elongate member 24. Electrical wire 72 is connected on a distal end thereof (not shown) to ablation electrode 16, and on a proximal end thereof to an electrical connector 74, e.g. an electrical plug. Ablation electrode 16 is electrically coupled via electrical wire 72 to a positive terminal of an external power source (not shown). A ground electrode, connected to a negative terminal of the power source, may be attached onto the back of the subject, such as to close an electrical conduction pathway passing through the body of the subject (all not shown). In particular, the ground electrode may be placed such that the electrical conduction pathway passes through the target tissue. When a potential difference is established between the terminals of the power source, electrical current flows from ablation electrode 16 to the ground electrode, via the target tissue, thereby ablating the target tissue.

According to some embodiments, catheter handle 20 may include additional ports 82, for example, for introducing fluids directly into the outlet channel. In some embodiments, the inlet channel and/or the outlet channel may function as a delivery catheter, and one or more of additional ports 82 may be configured for introducing a catheter tube into the inlet channel and/or the outlet channel. In some of these embodiments, elongate member 24 does not include either the inlet channel or the outlet channel.

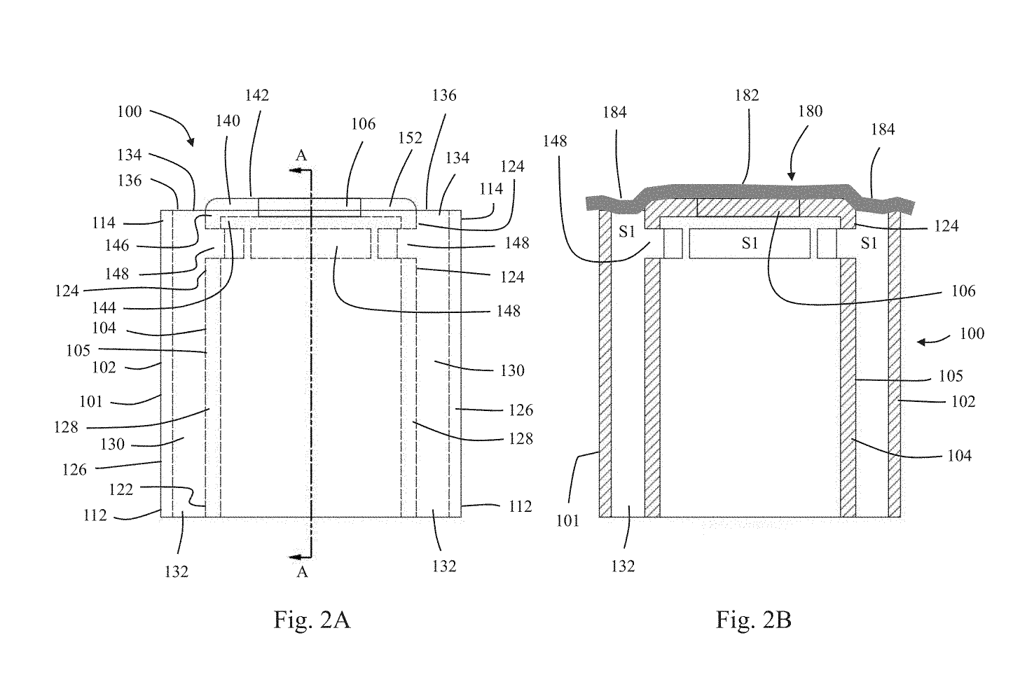

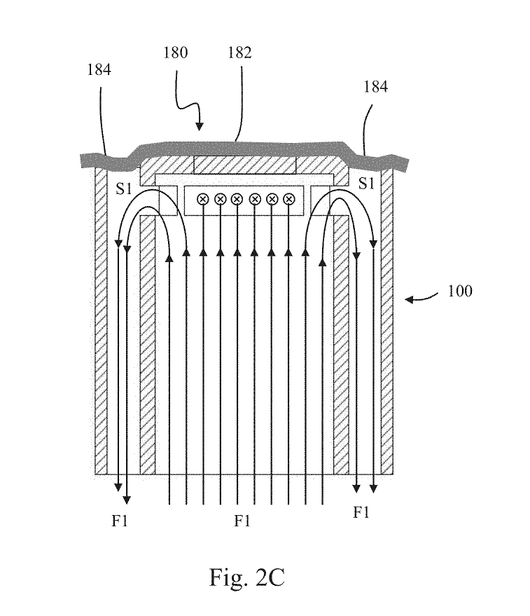

An exemplary embodiment of an ablation catheter tip 100, as described herein, is schematically depicted in FIGS. 2A-2D. As seen in FIG. 2A, ablation catheter tip 100 includes a tip body 101 in the form of a tubular member 102. Ablation catheter tip 100 further includes an inlet channel 104 and an ablation electrode 106. Inlet channel 104 is embodied by a tube 105. Tubular member 102 extends from a proximal member end 112 to a distal member end 114. Inlet channel 104 extends from a proximal inlet channel end 122, which is open and located at proximal member end 112, to a distal inlet channel end 124, located at distal member end 114. Inlet channel 104 is longitudinally disposed within tubular member 102. FIG. 2B schematically depicts a cross-sectional view of ablation catheter tip 100 taken along a line A-A (indicated in FIG. 2A), with a target tissue 180 attached to ablation catheter tip 100, as elaborated on hereinbelow.

According to some embodiments, tubular member 102 and inlet channel 104 are cylindrical. According to some embodiments, tubular member 102 and/or inlet channel 104 have, for example, a hexagonal or an octagonal cross-section perpendicularly to line A-A. According to some embodiments, inlet channel 104 is concentrically disposed within tubular member 102.

Tubular member walls 126 (that is to say, the walls of tubular member 102, which extend from proximal member end 112 to distal member end 114) and inlet channel walls 128 (that is to say, the walls of inlet channel 104, which extend from proximal inlet channel end 122 to distal inlet channel end 124) define an outlet channel 130. Outlet channel 130 includes the space inside tubular member 102, which is outside of inlet channel 104. Outlet channel 130 extends from a proximal outlet channel end 132, located at proximal member end 112, to a distal outlet channel end 134, located at distal member end 114.

Tubular member 102 includes a suction port 136 at distal member end 114. Suction port 136 surrounds distal inlet channel end 124. Suction port 136 is fluidly connected to outlet channel 130 via distal outlet channel end 134. Suction port 136 functionality is elaborated on hereinbelow.

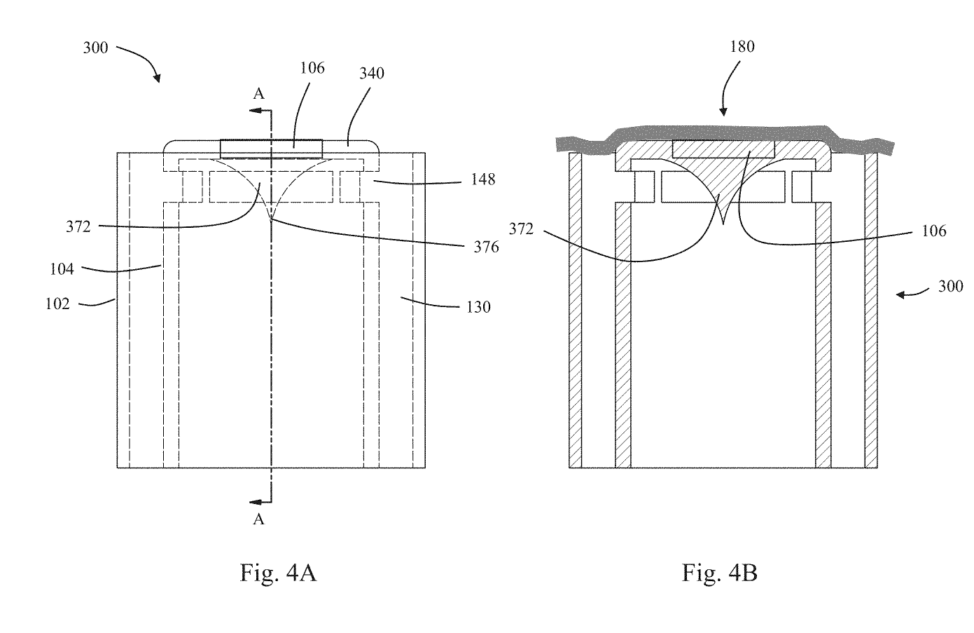

Inlet channel 104 includes an inlet channel cap 140 at distal inlet channel end 124. According to some embodiments, inlet channel cap 140 is disc-like and is mounted perpendicularly to line A-A. Inlet channel cap 140 includes an external cap surface 142 (that is to say, the surface of inlet channel cap 140 which is exposed on the outside of catheter tip 100). Inlet channel cap 140 further includes an internal cap surface 144 (that is to say, the surface of inlet channel cap 140 which is exposed within ablation catheter tip 100 at distal inlet channel end 124) and a cap edge 146, extending along the circumference of inlet channel cap 140 (and surround by suction port 136). According to some embodiments, external cap surface 142 is flat or convex Similarly, internal cap surface 144 may be flat or convex. According to some embodiments, the internal cap surface may be conic, extending proximally inside inlet channel 104, as shown in FIGS. 4A-4B.

Inlet channel 104 further includes fluid openings 148. According to some embodiments, fluid openings 148 are annularly disposed about distal inlet channel end 124. Fluid openings 148 fluidly connect inlet channel 104 to outlet channel 130. Apart from fluid connectivity via fluid openings 148 and proximal inlet channel end 122, inlet channel 104 is fluidly sealed.

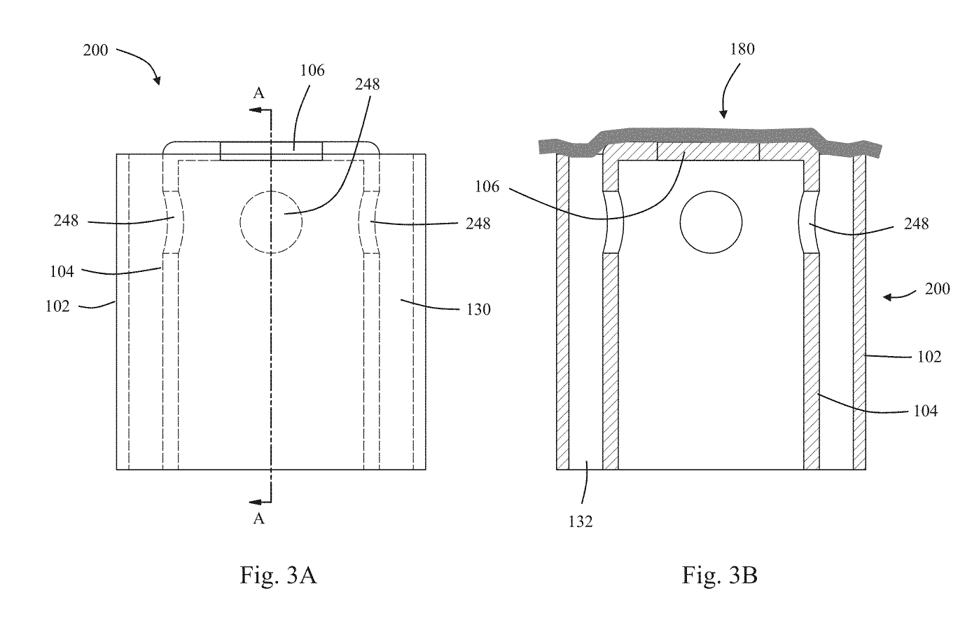

According to some embodiments, fluid openings 148 are oblong. According to some embodiments, as shown in FIGS. 3A-3B, the fluid openings may be round. According to some embodiments, fluid openings 148 consist of a single opening. According to some embodiments, fluid openings 148 consist of two, three, four, or even ten or twenty openings.

Ablation electrode 106 is positioned on/in inlet channel cap 140, such as to be at least partially exposed on external cap surface 142. In some embodiments ablation electrode 106 is embedded on/in inlet channel cap 140. In some embodiments ablation electrode 106 is attached onto inlet channel cap 140. In some embodiments ablation electrode 106 is integrally formed with inlet channel cap 140. In some embodiments ablation electrode 106 is additionally exposed on internal cap surface 144. In some embodiments inlet channel cap 140 includes a cap portion 152 adjacent to ablation electrode 106. In some embodiments, ablation electrode 106 is not exposed on internal cap surface 144, cap portion 152 is at least partially exposed on internal cap surface 144, and is further a good heat conductor. In some embodiments, ablation electrode 106 is cylindrical with a radius between about 1 mm and about 1.5 mm and a length between about 2 mm and about 12 mm In some embodiments, ablation electrode 106 may be made of platinum-iridium or of gold.

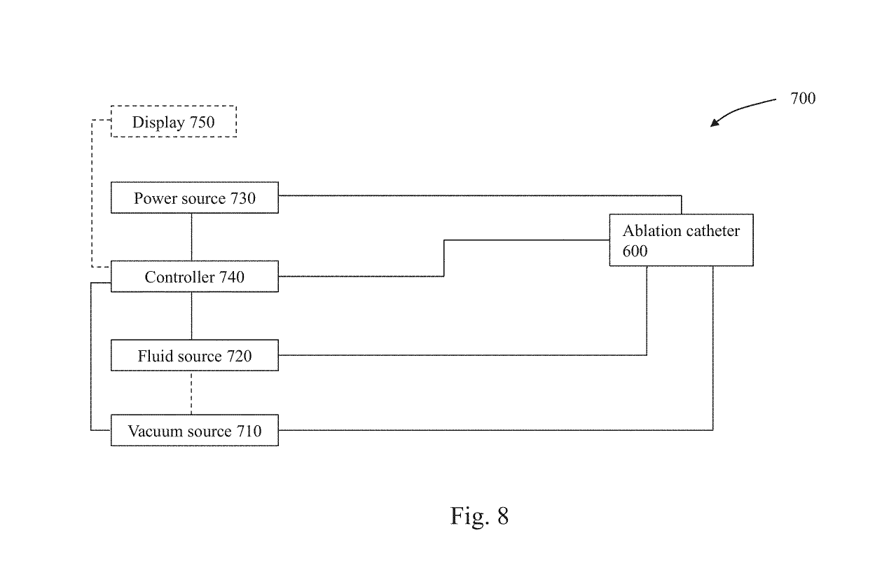

A temperature sensor (not shown) is also positioned in/on inlet channel cap 140. Additional sensors, such as a force sensor (not shown) for gauging the strength of the attachment of a target tissue to ablation electrode 106, for example, by measuring the bending of optical fibers, as in the TactiCath™ sensor by Endosense SA, and a pressure sensor (not shown), e.g. for measuring the pressure at suction port 136, may be positioned in/on in inlet channel cap 140, tubular member walls 126, and/or on inlet channel walls 128. Further, mapping and sensing electrodes (electric activity measuring electrodes) for determining the location of the target tissue, imaging sensors to help guide ablation catheter tip 100, such as a CCD camera (not shown), and a light source (not shown), may be positioned in/on inlet channel cap 140, tubular member walls 126, and/or on inlet channel walls 128. The functionality of the above-mentioned sensors is elaborated on hereinbelow. Electrical wires (not shown) extend through inlet channel 104, outlet channel 130, tubular member walls 126, and/or inlet channel walls 128. The electrical wires supply power to ablation electrode 106, and may also supply power to some or all of the sensors. Data transmission wires (not shown), e.g. electrical wires and/or optical fiber cables, further transmit sensed readings, e.g. temperature readings by the temperature sensor, and/or images from the imaging sensors, respectively, to an external control circuitry, as described in the description of FIG. 8. Optionally, the data transmission wires may further transmit instructions from the external control circuitry to some or all of the sensors.

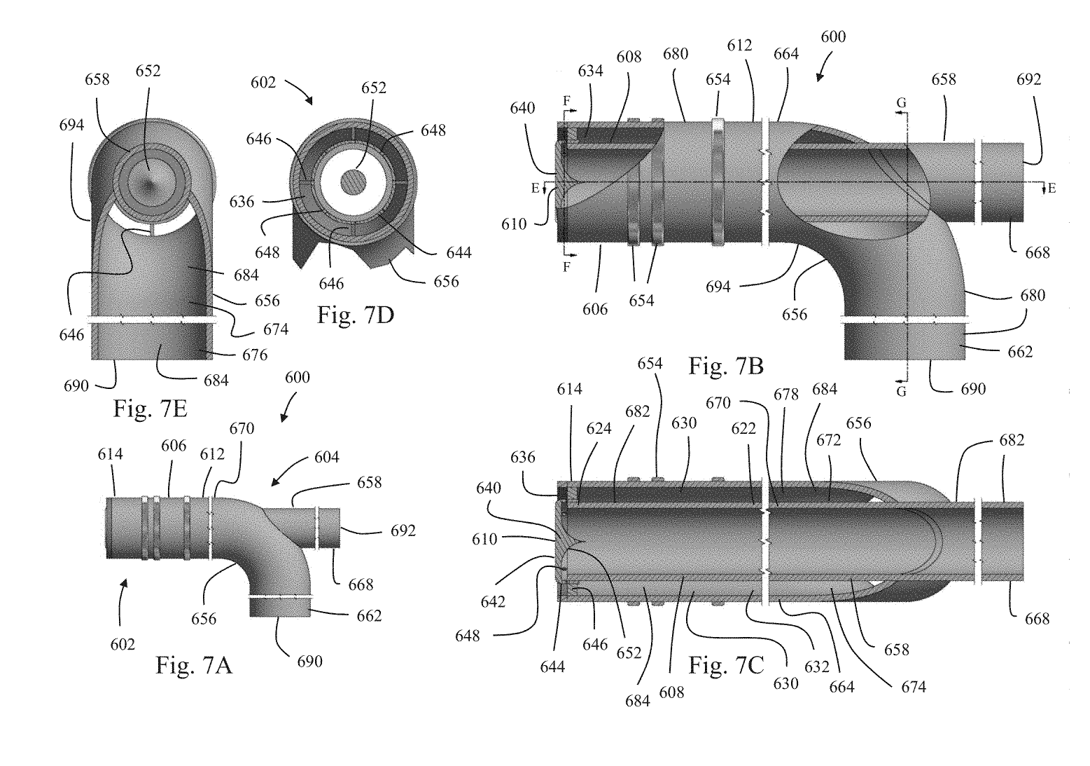

According to some embodiments, ablation catheter tip 100 is configured to be detachably mounted on a catheter tubing assembly. According to some embodiments, ablation catheter tip 100 is not detachable, forming an integral part of the catheter tubing assembly. These options are elaborated on in the description of FIGS. 7A-7E.

FIGS. 2B-2C schematically depicts ablation catheter tip 100 in operation, as used in treating AF, according to some embodiments: Ablation catheter tip 100 is guided into the left atrium chamber proximately to a pulmonary vein opening, as described in the description of FIG. 1. In some embodiments, the mapping and sensing electrodes may be used to identify target tissue 180, and determine a location thereof on the pulmonary vein opening, as elaborated on hereinbelow. In some embodiments, an electro-sensor catheter is inserted into the left atrium chamber to measure electrical activity and thereby identify target tissue 180. The position and orientation of ablation catheter tip 100 is adjusted to bring ablation electrode 106 sufficiently near a tissue ablation site 182 on target tissue 180, facing tissue ablation site 182, such that when suction is applied via suction port 136 (that is to say, when a pressure lower than blood pressure is induced at suction port 136), ablation electrode 106 is secured to tissue ablation site 182. Suction port 136 and an adjacent tissue 184, surrounding tissue ablation site 182, are drawn towards each other. Suction port 136 is thereby covered by adjacent tissue 184 and blocked, and a closed (or effectively closed) irrigation zone S1 is formed. Closed irrigation zone S1 includes the space about distal member end 114, which is fluidly disconnected from the left atrium chamber by target tissue 180 at tissue ablation site 182 and particularly by adjacent tissue 184.

Once ablation electrode 106 has been secured to target tissue 180 and closed, irrigation zone S1 has been sealed, a coolant (or in some embodiments, some other type of irrigant) is introduced into inlet channel 104 via proximal inlet channel end 122. The coolant flows until distal inlet channel end 124, wherefrom it is directed via fluid openings 148 into distal outlet channel end 134. Due to a vacuum force acting proximally along outlet channel 130 and inducing the suction at suction port 136, and the resultant blocking of suction port 136, substantially all of the coolant is made to proximally flow along outlet channel 130, exiting via proximal outlet channel end 132. Arrows F1 represent the coolant's flow direction. The symbol ⊗ represents a flow direction into the plane of the page. The coolant washes away blood in closed irrigation zone S1. In particular, the coolant will wash away ablation byproducts such as char.

Following the introduction of the coolant, the ablation is begun by applying a current, e.g. an RF current, through ablation electrode 106. Consequently, ablation electrode 106 and target tissue 180, particularly at tissue ablation site 182, as well as adjacent tissue 184, begin heating. The coolant is washed against internal cap surface 144, thereby cooling ablation electrode 106, that is to say, absorbing heat from ablation electrode 106. In embodiments wherein ablation electrode 106 is exposed on internal cap surface 144, the cooling via internal cap surface 144 is effected, at least in part, directly. In embodiments wherein ablation electrode 106 is not exposed on internal cap surface 144, the cooling via internal cap surface 144 is effected indirectly, with heat flowing from ablation electrode 106 to the coolant via cap portion 152.

As the coolant passes through fluid openings 148, some of the coolant washes against cap edge 146, thereby further cooling inlet channel cap 140, and consequently cooling ablation electrode 106 from the outside of inlet channel 104. Further, some of the coolant passing through fluid openings 148 may be washed and/or sprayed against adjacent tissue 184, thereby cooling adjacent tissue 184. The cooling of adjacent tissue 184 may contribute to the cooling of tissue ablation site 182, as well as to preventing heat from spreading to tissue beyond adjacent tissue 184.

It is noted that adjacent tissue 184 may be part of, or partially overlap with, target tissue 180, for example, when target tissue 180 includes more than a single tissue ablation site. That is to say, when target tissue 180 includes additional tissue ablation sites beyond tissue ablation site 182, such that at least some of the additional tissue ablation sites are adjacent to tissue ablation site 182, then there will be at least some overlap between the additional tissue sites and adjacent tissue 184.

It will be understood by the skilled person that the flow direction of the coolant may be reversed, such that the coolant is introduced via proximal outlet channel end 132 and expelled via proximal inlet channel end 122. In such embodiments, the suction is applied via inlet channel 104, that is to say, the vacuum force will act in the proximal direction along inlet channel 104.

According to some embodiments, ablation electrode 106 and a ground electrode (not shown) are arranged in bipolar configuration. That is to say, the ground electrode is also positioned at distal member end 114, for example, on inlet channel walls 128, or on cap portion 152 (instead of being located externally to the subject's body, as described in the description of ablation catheter 10).

FIGS. 3A-3B schematically depict an exemplary embodiment of an ablation catheter tip 200. As seen in FIG. 3A, ablation catheter tip 200 is essentially similar to catheter tip 100 except for including fluid openings 248 in place of fluid openings 148. Fluid openings 248 are essentially similar to fluid openings 148 except for being round rather than oblong. FIG. 3B schematically depicts a cross-sectional view of catheter tip 200 taken along line A-A, with target tissue 180 attached to ablation catheter tip 200.

FIGS. 4A-4B schematically depict an exemplary embodiment of an ablation catheter tip 300. As seen in FIG. 4A, ablation catheter tip 300 is essentially similar to catheter tip 100 except for including an inlet channel cap 340 in place of inlet channel cap 140. Inlet channel cap 340 differs from inlet channel cap 140 in additionally including a guide structure 372. Guide structure 372 is configured to help deflect, and thereby help direct, a distal flow of the coolant—arriving at distal inlet channel end 124 (via inlet channel 104)—into fluid openings 148 and thereby into outlet channel 130. According to some embodiments, guide structure 372 is conic, extending proximally from inlet channel cap 340 inside inlet channel 104, and ending in a cone tip 376. According to some embodiments, guide structure 372 is shaped as a concave cone. FIG. 4B schematically depicts a cross-sectional view of catheter tip 300 taken along line A-A, with target tissue 180 attached to ablation catheter tip 300.

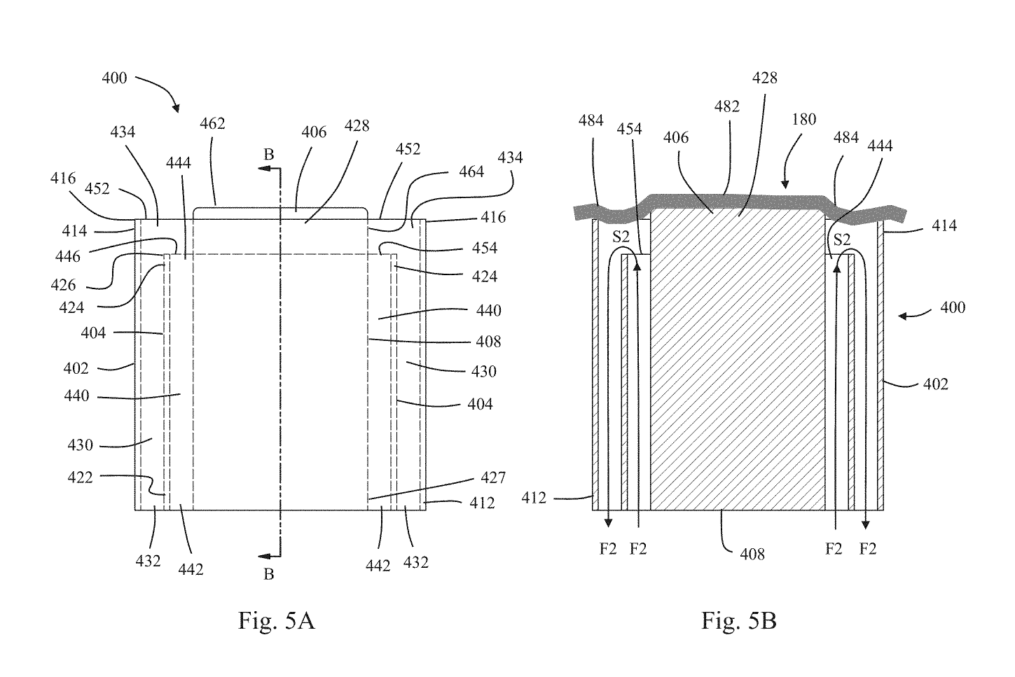

FIGS. 5A-5C schematically depict an exemplary embodiment of an ablation catheter tip 400. As seen in FIG. 5A, ablation catheter tip 400 includes a tubular member 402, an inlet tube 404, an ablation electrode 406, and an inner core 408. Tubular member 402, is similar to tubular member 102, extending from a proximal member end 412 to a distal member end 414. A distal member extremity 416 consists of the distal edge of distal member end 414. Inlet tube 404 extends from a proximal tube end 422, which is open and located at proximal member end 412, to a distal tube end 424, which is open and located at distal member end 414. Inlet tube 404 is longitudinally disposed within tubular member 402. A distal tube extremity 426 consists of the distal edge of distal inlet tube end 424. Inlet tube 404 extends slightly less in the distal direction than tubular member 402, that is to say, inlet tube 404 does not distally extend until distal member extremity 416.

Inner core 408 is longitudinally disposed within inlet tube 404. Inner core 408 extends from a proximal core end 427, located at proximal tube end 422, to a core tip 428, which distally extends beyond distal tube extremity 426, as elaborated on hereinbelow. According to some embodiments, tubular member 402, inlet tube 404, and inner core 408 are all cylindrical and are concentrically disposed. FIG. 5B schematically depicts a cross-sectional view of ablation catheter tip 400 taken along a line B-B (indicated in FIG. 5A).

Similarly to tubular member 102 and inlet channel 104 in ablation catheter tip 100, tubular member 402 and inlet tube 404 define an outlet channel 430. Outlet channel 430 includes the space within tubular member 402, which is outside of inlet tube 404. Outlet channel 430 extends from a proximal outlet channel end 432, located at proximal member end 412, to a distal outlet channel end 434, located at distal member end 414. Similarly, inlet tube 404 and inner core 408 define an inlet channel 440. Inlet channel 440 extends from a proximal inlet channel end 442, located at proximal tube end 422, to a distal inlet channel end 444, located at distal tube end 424. A distal inlet channel extremity 446 is located at distal tube extremity 426.

A suction port 452 is located at distal member extremity 416, circumscribing inner core 408. Suction port 452 is fluidly connected to outlet channel 430 via distal outlet channel end 434. A fluid opening 454 is located at distal tube extremity 426, circumscribing inner core 408. Fluid opening 454 fluidly connects distal inlet channel end 444 to distal outlet channel end 434 (thereby fluidly connecting inlet channel 440 to outlet channel 430).

According to some embodiments, core tip 428 is flat or convex. According to some embodiments, core tip 428 extends slightly further in the distal direction than distal member extremity 416. Ablation electrode 406 is positioned on/in core tip 428 such as to be at least partially exposed on a top core surface 462 of core tip 428. According to some embodiments, ablation electrode 406 may also be at least partially exposed on a circumferential core surface 464, which circumscribes core tip 428. According to some embodiments, circumferential core surface 464 is made of a material having a high heat conductance. According to some embodiments, circumferential core surface 464 is indirectly thermally coupled to ablation electrode 406 via a core tip internal portion (not indicated), which is adjacent to ablation electrode 406 and which is a good heat conductor. According to some embodiments, circumferential core surface 464 is directly thermally coupled to ablation electrode 406, e.g. ablation electrode 406 is exposed on circumferential core surface 464.

A temperature sensor (not shown) is also positioned on/in core tip 428. Additional sensors, components, and electrical and data transmission wires, as listed above in the description of catheter tip 100, may be positioned on/in core tip 428, or elsewhere along inner core 408, on inlet tube 404, and/or on tubular member 402.

Ablation catheter tip 400 is operated similarly to ablation catheter tip 100, and the following description of ablation catheter tip 400 operation may be complemented by referring to the description of ablation catheter tip 100 operation hereinabove. FIG. 5B schematically depicts ablation catheter tip 400 in operation. A vacuum force exerted via suction port 452 secures core tip 428 onto a tissue ablation site 482 on target tissue 180. Suction port 452 is blocked by an adjacent tissue 484, which surrounds tissue ablation site 482. Since inlet tube 404 does not extend in the distal direction as much as tubular member 402 and inner core 408, inlet channel 440 remains fluidly connected to outlet channel 430 via fluid opening 454, even when suction port 452—being located at distal member extremity 416—is blocked. A closed irrigation zone S2, similar to closed irrigation zone S1, is thereby formed and fluidly sealed. The coolant washes away blood in closed irrigation zone S2. In particular, the coolant will wash away ablation byproducts, such as char, and/or prevent or reduce the formation of ablation byproducts.

Following the securing of ablation electrode 406 to target tissue 180 and the blocking of suction port 452, a coolant is introduced via proximal inlet channel end 442. The coolant distally flows through inlet channel 440 to distal inlet channel end 444, wherefrom the coolant is directed via fluid opening 454 into distal outlet channel 434. The coolant flows proximally through outlet channel 430 and exits via proximal outlet channel end 432. Arrows F2 represent the coolant's flow direction.

As the coolant passes through fluid opening 454, some of the coolant washes circumferential core surface 464, thereby cooling ablation electrode 406. Further, some of the coolant passing through fluid opening 454 may be washed and/or sprayed against adjacent tissue 484, thereby cooling adjacent tissue 484. The cooling of adjacent tissue 484 may contribute to the cooling of tissue ablation site 482, as well as to preventing heat from spreading to tissue beyond adjacent tissue 484.

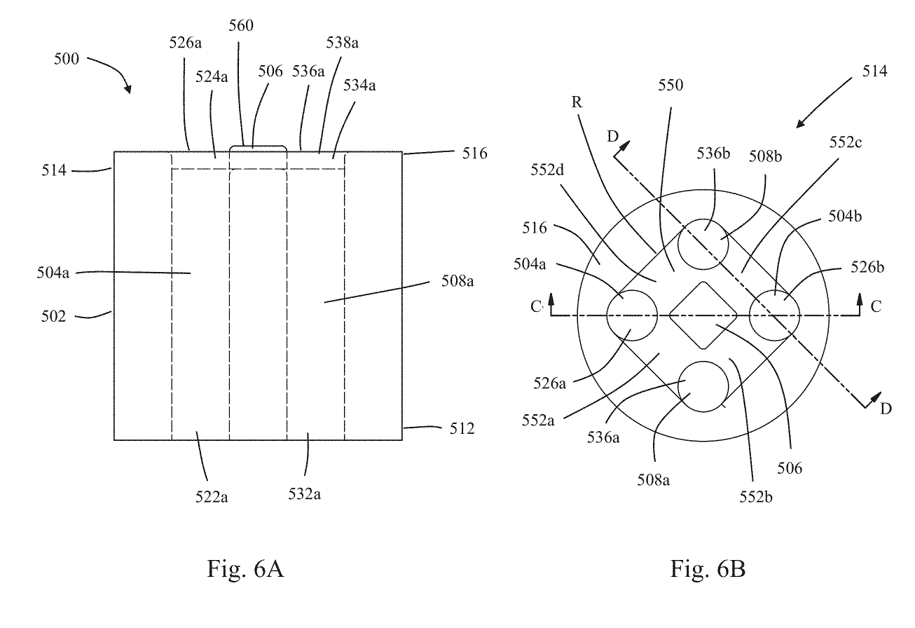

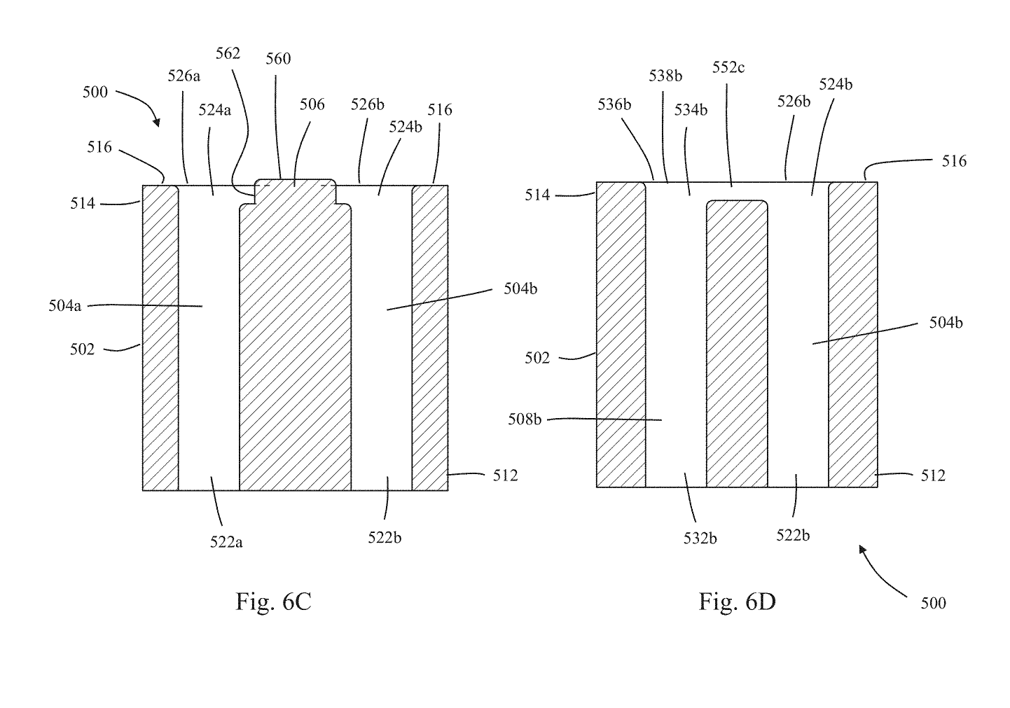

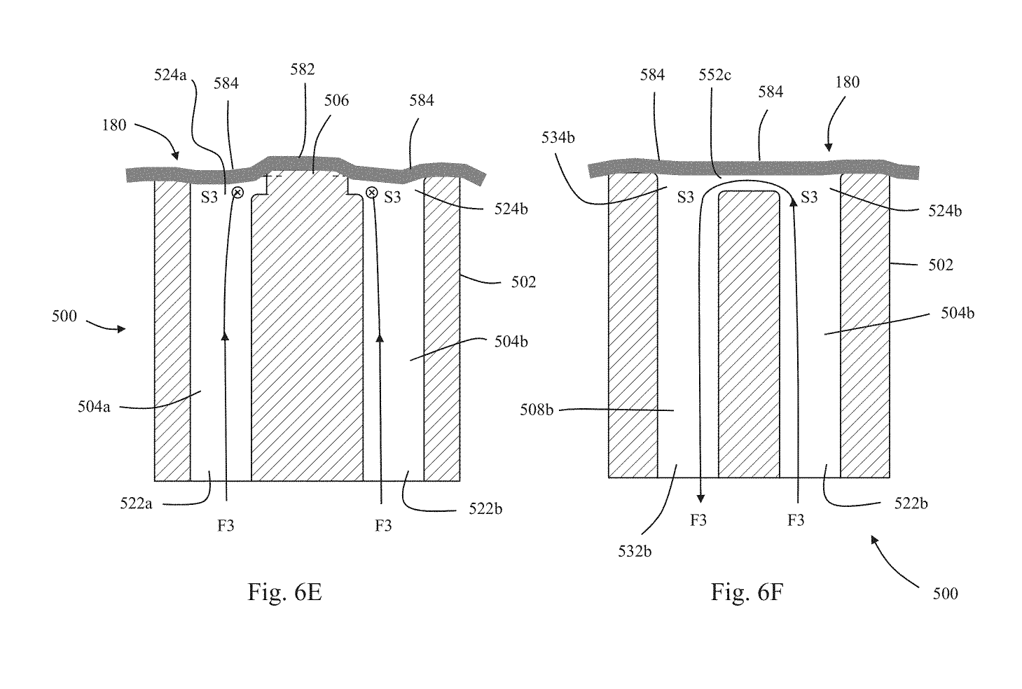

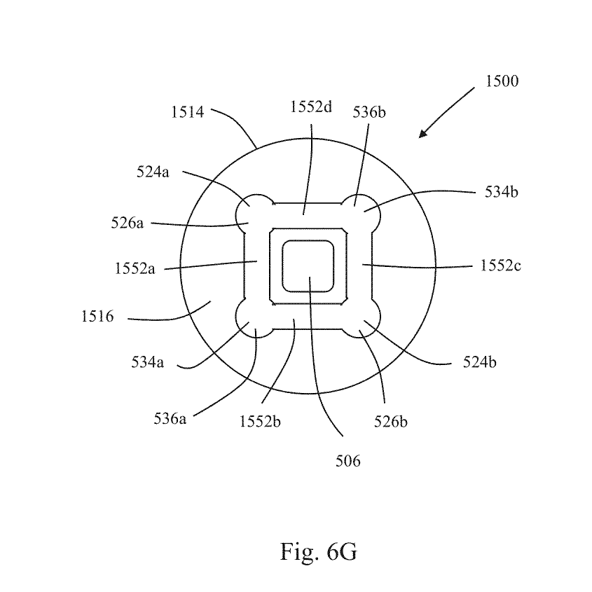

FIGS. 6A-6F schematically depict an exemplary embodiment of an ablation catheter tip 500. FIG. 6A provides a side-view of ablation catheter tip 500, and FIG. 6B provides a top view thereof. Ablation catheter tip 500 includes a tip body 502, a first inlet channel 504a, a second inlet channel 504b (shown in FIGS. 6B-6D), an ablation electrode 506, a first outlet channel 508a, and a second outlet channel 508b (shown in FIGS. 6B and 6D). Tip body 502 extends from a proximal tip body end 512 to a distal tip body end 514. Ablation electrode 506 is positioned at distal tip body end 514. A distal tip body extremity 516 consists of the distal edge of distal tip body end 514.

Each of inlet channels 504a and 504b is tubular and is longitudinally disposed within tip body 502. First inlet channel 504a extends from a proximal first inlet channel end 522a, located at proximal tip body end 512, to a distal first inlet channel end 524a, located at distal tip body end 514 Similarly, second inlet channel 504b extends from a proximal second inlet channel end 522b, located at proximal tip body end 512, to a distal second inlet channel end 524b, located at distal tip body end 514. A distal first inlet channel extremity 526a consists of the distal edge of first inlet channel 504a, and coincides with distal tip body extremity 516. A distal second inlet channel extremity 526b consists of the distal edge of second inlet channel 504b, and coincides with distal tip body extremity 516.

Each of outlet channels 508a and 508b is tubular and is longitudinally disposed within tip body 502. First outlet channel 508a extends from a proximal first outlet channel end 532a, located at proximal tip body end 512, to a distal first outlet channel end 534a, located at distal tip body end 514. Similarly, second outlet channel 508b extends from a proximal second outlet channel end 532b, located at proximal tip body end 512, to a distal second outlet channel end 534b, located at distal tip body end 514. A distal first outlet channel extremity 536a consists of the distal edge of first outlet channel 508a, and coincides with distal tip body extremity 516. A distal second outlet channel extremity 536b consists of the distal edge of second outlet channel 508b, and coincides with distal tip body extremity 516. A first suction port 538a is mounted at distal first outlet channel extremity 536a (and is fluidly connected to distal first outlet channel end 534a). A second suction port 538b is mounted at distal second outlet channel extremity 536b (and is fluidly connected to distal second outlet channel end 534b).

FIG. 6B schematically depicts a top view of distal tip body end 514. As seen in FIG. 6B, inlet channels 504a and 504b and outlet channels 508a and 508b are arranged in a square configuration, defining a square R. First inlet channel 504a and second inlet channel 504b are located on opposite corners of square R. Similarly, first outlet channel 508a and second outlet channel 508b are also located on opposite corners of square R. Ablation electrode 506 is located in the center of square R. FIG. 6C schematically depicts a cross-sectional view of ablation catheter tip 500 taken along a line C-C, which bisects inlet channels 504a and 504b. FIG. 6D schematically depicts a cross-sectional view of ablation catheter tip 500 taken along a line D-D, which bisects second inlet channel 504b and second outlet channel 508b.

As shown in FIG. 6B-6D, a recessed region 550 surrounds ablation electrode 506 and forms a depression into distal tip body end 514 from distal tip body extremity 516. Recessed region 550 is divided into four recessed sub-regions: a recess 552a, a recess 552b, a recess 552c, and a recess 552d. As shown in FIG. 6D, recess 552c maintains second inlet channel 504b and second outlet channel 508b fluidly connected when distal second inlet channel extremity 526b and distal second outlet channel extremity 536b (i.e. second suction port 538b) are blocked (and recess 552c is blocked on distal tip body extremity 516) Similarly, recesses 552a, 552b, and 552d, fluidly connect first inlet channel 504a to first outlet channel 508a, first outlet channel 508a to second inlet channel 504b, and second outlet channel 508b to first inlet channel 504a, respectively.

As shown in FIG. 6C, ablation electrode 506 includes an external electrode surface 560 on the distal edge thereof, and an electrode edge 562, circumscribing ablation electrode 506. External electrode surface 560 location is distal relative to distal tip body extremity 516, that is to say, ablation electrode 506 extends slightly beyond distal tip body extremity 516 in the distal direction. According to some embodiments, external electrode surface 560 is flat or convex.

A temperature sensor (not shown) is positioned on/in distal tip body end 514.

Additional sensors, components, and electrical and data transmission wires, as listed above in the description of catheter tip 100, may be positioned on/in distal tip body end 514, or along tip body 502.

Ablation catheter tip 500 is operated similarly to ablation catheter tip 100, and the following description of ablation catheter tip 500 operation may be complemented by referring to the description of ablation catheter tip 100 operation hereinabove. FIGS. 6E-6F schematically depict ablation catheter tip 500 in operation. Suctions exerted at first suction port 538a and second suction port 538b secure ablation electrode 506 onto a tissue ablation site 582 on target tissue 180. Suction ports 538a and 538b, distal inlet channel extremities 526a and 526b, and recesses 552a-552d are covered by an adjacent tissue 584, around tissue ablation site 582. A closed irrigation zone S3 is thereby formed and fluidly sealed. Closed irrigation zone S3 includes distal inlet channel ends 524a and 524b, distal outlet channel ends 534a and 534b (including suction ports 538a and 538b), and recesses 552a-552d.

As shown in FIGS. 6C and FIG. 6E, ablation electrode 506 position, particularly, ablation electrode 506 distal extension beyond distal tip body extremity 516, obstructs fluid communication between distal first inlet channel end 524a and distal second inlet channel end 524b via closed irrigation zone S3. Similarly, ablation electrode 506 mounting position obstructs fluid communication between distal first outlet channel end 534a and distal second outlet channel end 534b via closed irrigation zone S3.

As shown in FIGS. 6E-6F, following the securing of ablation electrode 506 to target tissue 180 and the forming of closed irrigation zone S3, a coolant is introduced via proximal inlet channel ends 522a and 522b. The coolant distally flows through inlet channels 504a and 504b, respectively, to distal inlet channel ends 524a and 524b. From each of distal inlet channel ends 524a and 524b, the coolant flows into both of distal outlet channel ends 534a (not shown in FIGS. 6E-6F) and 534b, respectively, due to the suction exerted at suctions ports 538a and 538b, respectively. For example, some of the coolant arriving at distal second inlet channel end 524b is directed via recess 552c into distal second outlet channel end 534b. The remainder of the coolant arriving at distal second inlet channel end 524b is directed via recess 552b (not shown in FIGS. 6E-6F) into distal first outlet channel end 534a. The coolant flows proximally through outlet channels 508a and 508b, exiting via proximal outlet channel ends 532a and 532b, respectively. Arrows F3 represent the coolant's flow direction. The coolant washes away blood in closed irrigation zone S3. In particular, the coolant will wash away ablation byproducts such as char.

As the coolant passes through distal channel ends 524a, 524b, 534a, and 534b and recesses 552a-552d, some of the coolant washes electrode edge 562, thereby cooling ablation electrode 506. Further, some of the coolant may wash against adjacent tissue 584, thereby cooling adjacent tissue 584. The cooling of adjacent tissue 584 may contribute to the cooling of tissue ablation site 582, as well as to preventing heat from spreading to tissue beyond adjacent tissue 584.

FIG. 6G schematically depict a top view of an exemplary embodiment of an ablation catheter tip 1500. Ablation catheter tip 1500 is essentially similar to ablation catheter tip 500 except for including a distal tip body end 1514 in place of distal tip body end 514. Distal tip body end 1514 includes recesses 1552a, 1552b, 1552c, and 1552d in place of recesses 552a-552d, respectively, but is otherwise similar to distal tip body end 514. Recess 1552a forms an oblong depression into distal tip body end 1514 from a distal tip body extremity 1516 (the distal edge of distal tip body end 1514) and fluidly connects distal first inlet channel end 524a to distal first outlet channel end 534a. Recess 1552a maintains distal first inlet channel end 524a and distal first outlet channel end 534a fluid connectivity when distal first inlet channel extremity 526a and distal first outlet channel extremity 536a are blocked and recess 1552a is blocked on distal tip body extremity 1516 (e.g. by tissue, essentially as described in the description of the operation of ablation catheter tip 500). Similarly, recess 1552b fluidly connects distal first outlet channel end 534a to distal second inlet channel end 524b, recess 1552c fluidly connects distal second inlet channel end 524b to distal second outlet channel end 534b, and recess 1552d fluidly connects distal second outlet channel end 534b to distal first inlet channel end 524a.

Each of ablation catheter tips 100, 200, 300, 400, 500, and 1500 provides a different exemplary embodiment of ablation catheter tip 14.

FIGS. 7A-7E schematically depict an embodiment of an ablation catheter 600, including an ablation catheter tip 602 and a catheter tubing assembly 604. As seen in FIGS. 7A-7B, ablation catheter tip 602 is mounted on catheter tubing assembly 604, as elaborated on hereinbelow. FIG. 7C schematically depicts a cross-sectional view of ablation catheter 600 taken along a line E-E (indicated in FIG. 7B). FIG. 7D schematically depicts a cross-sectional view of ablation catheter tip 602 taken along a line F-F (indicated in FIG. 7B). FIG. 7D schematically depicts a cross-sectional view of catheter tubing assembly 604 taken along a line G-G (indicated in FIG. 7B).

As shown in FIGS. 7A-7D, ablation catheter tip 602 includes a tubular member 606, an inlet channel 608, and an ablation electrode 610. Tubular member 606 extends from a proximal member end 612 to a distal member end 614. Inlet channel 608 is tubular and is longitudinally disposed within tubular member 606. Inlet channel 608 extends from a proximal inlet channel end 622, which is open and located at proximal member end 612, to a distal inlet channel end 624, located at distal member end 614.

Similarly to tubular member 102 and inlet channel 104 of ablation catheter tip 100, tubular member 606 and inlet channel 608 define an outlet channel 630. Outlet channel 630 includes the space within tubular member 606, which is outside of inlet channel 608. Outlet channel 630 extends from a proximal outlet channel end 632, located at proximal member end 612, to a distal outlet channel end 634, located at distal member end 614. Tubular member 606 includes a suction port 636 at distal member end 614. Suction port 636 is fluidly connected to outlet channel 630 via distal outlet channel end 634.

An inlet channel cap 640 is mounted at distal inlet channel end 624. Inlet channel cap 640 includes a cap top 642 and a cap edge 644. Cap edge 644 circumscribes distal inlet channel end 624, and is surrounded by suction port 636. Ablation electrode 610 is mounted on inlet channel cap 640, essentially similarly to how ablation electrode 106 is mounted on inlet channel cap 140.

Supports 646 are located at distal channel end 624. Each of supports 646 extends radially from inlet channel 608 to tubular member 606. According to some embodiments, cap edge 644 includes holes (not indicated in the Figures) circumferentially disposed thereon, and each of supports 646 extends through a respective hole (of the holes), thereby securing inlet channel cap 640 to distal inlet channel end 624.

According to some embodiments, supports 646 help secure inlet tube 608 to tubular member 606. Further supports, similar to supports 646, may be positioned along inlet channel 608, for example, proximately to proximal inlet channel end 622, and/or midway between proximal inlet channel end 622 and distal inlet channel end 624.

Cap edge 644 includes fluid openings 648, which are annularly disposed thereon. Fluid openings 648 fluidly connect inlet channel 608 to outlet channel 630 in an essentially similar manner to the fluid connection between inlet channel 104 and outlet channel 130 provided by fluid openings 148. According to some embodiments, inlet channel cap 640 includes a guide structure 652, essentially similar to guide structure 372.

A temperature sensor (not shown) is positioned on/in inlet channel cap 640. Additional sensors, components, and electrical and data transmission wires, as listed above in the description of ablation catheter tip 100, may be positioned on/in inlet channel cap 640, and/or elsewhere along inlet channel 608 and/or outlet channel 630.

In some embodiments, tubular member 606 includes mapping and sensing electrode rings 654, which are annularly disposed thereon. Mapping and sensing electrode rings 654 are configured to sense atrial electrical signals. The sensed electrical signals are sent to an external processor (e.g. in controller 740 in FIG. 8) for analysis, which is used to determine the location of the target tissue. According to some embodiments, mapping and sensing electrode rings 654 are further configured to transmit electrical signals and to sense resultant electrical signals reflected off the walls of the left atrium (or off the walls of any other body cavity).

As shown in FIGS. 7A-7C and FIG. 7E, catheter tubing assembly 604 includes a tubular member extension 656 and an inlet channel extension 658, which are both flexible. Tubular member extension 656 extends from a proximal member extension end 662 to a distal member extension end 664. Inlet channel extension 658 extends from a proximal inlet channel extension end 668 to a distal inlet channel extension end 670, located at distal member extension end 664. An inlet channel extension distal portion 672 (i.e. a distal portion of inlet channel extension 658) is located within tubular member extension 656 and is longitudinally disposed therein.

Tubular member extension 656 and inlet channel extension 658 define an outlet channel extension 674. Outlet channel extension 674 includes the space within tubular member extension 656, which is outside of inlet channel extension 658. Outlet channel extension 674 extends from a proximal outlet channel extension end 676 (shown in FIG. 7E) to a distal outlet channel extension end 678 (shown in FIG. 7C).

According to some embodiments, catheter tip 602 and catheter tubing assembly 604 form an integral structure, that is to say, ablation catheter 600 is integrally formed. Distal inlet channel extension end 670 is joined to proximal inlet channel end 622, thereby fluidly connecting proximal inlet channel extension end 668 to distal inlet channel end 624. Distal member extension end 664 is joined to proximal member end 612, such as to fluidly connect outlet channel 630 to outlet channel extension 674 (in particular, fluidly connecting proximal outlet channel extension end 676 to distal outlet channel end 634).

An extended tubular member 680 includes tubular member extension 656 and tubular member 606, extending from proximal member extension end 662 to distal member end 614. An extended inlet channel 682 includes inlet channel extension 658 and inlet channel 608, extending from proximal inlet channel extension end 668 to distal inlet channel end 624. An extended outlet channel 684 includes outlet channel extension 674 and outlet channel 630, extending from proximal outlet channel extension end 676 to distal outlet channel end 634.

A vacuum port 690 is mounted on proximal member extension end 662, such as to be fluidly connected to proximal outlet channel extension end 676 and thereby to extended outlet channel 684. Vacuum port 690 is configured to be coupled to a vacuum source (not shown), e.g. a vacuum pump, and thereby to apply suction, via extended outlet channel 684, at suction port 636. A fluid inlet port 692 is mounted on proximal inlet channel extension end 668. Fluid inlet port 692 is configured for introducing a fluid, such as a coolant, into extended inlet channel 682. In some embodiments, one or more additional ports (not shown) may be mounted on proximal member extension end 662 and/or on proximal inlet channel extension end 668. In particular, an additional port (not shown) may be fluidly coupled to extended outlet channel 684. The additional port may be used to help adjust and fix the fluid pressure at suction port 636 to slightly above blood pressure, as elaborated on hereinbelow.

According to some embodiments, catheter tip 602 is detachably mountable on catheter tubing assembly 604.

In embodiments wherein inlet channel extension 658 is only partially disposed within tubular member extension 656 (i.e. only inlet channel extension distal portion 672 is disposed within tubular member extension 656), catheter tubing assembly 604 includes a tubing junction 694. Inlet channel extension 658 enters tubular member extension 656 at tubing junction 694, that is to say, inlet channel extension distal portion 672 extends distally from tubing junction 694. According to some embodiments, tubing junction 694 may be disposed within a catheter handle (not shown), such as catheter handle 20 in FIG. 1. According to some embodiments, tubing junction 694 may be located proximally relative to a catheter handle, with a short portion of catheter tubing assembly 604 being disposed within the catheter handle. FIG. 7E depicts a cross-sectional view in the distal direction of ablation catheter 600 taken along line G-G.

FIG. 8 schematically depicts a block diagram of an ablation setup 700 in accordance with the embodiments of the present disclosure. For simplicity, ablation setup 700 is described with reference to ablation catheter 600 (as shown in FIGS. 7A-7E). However, the skilled person will understand that ablation setup 700 may be implemented using ablation catheters other than ablation catheter 600, such as ablation catheter 10, and particularly ablation catheters including a catheter tip, such as catheter tip 100, 200, 300, 400, 500 or similar thereto. Ablation setup 700 includes ablation catheter 600, a vacuum source 710 for generating suction at suction port 636, a fluid source 720 for introducing fluid at a controllable introduction temperature into catheter 600, and a power source 730 for inducing an electrical current via ablation electrode 610. Ablation setup 700 further includes a controller 740 for coordinating and controlling functions of the above-listed components of ablation setup 700, as elaborated on hereinbelow. Optionally, ablation setup 700 further includes a display 750.

Vacuum source 710 includes a means for generating suction (not shown)—such as a vacuum pump, a hospital vacuum port, a fluid pump, or any liquid handling sub-pressure device—configured to allow varying the suction strength. Vacuum source 710 is controllably fluidly coupled to vacuum port 690, and thereby to extended outlet channel 684. By activating vacuum source 710 a force, acting in the proximal direction, is induced along extended outlet channel 684, and suction is applied at suction port 636. Vacuum source 710 further includes a drain (not shown), for expelling fluids arriving at vacuum source 710 via vacuum port 690.

Fluid source 720 is fluidly coupled to fluid inlet port 692, and is configured to introduce fluid—for example, by means of a fluid pump (e.g. a peristaltic pump), an elevated saline bag/container, or any other saline flow control system (all not shown)—into fluid inlet port 692, and thereby into extended inlet channel 682. Fluid source 720 includes a fluid flow modulator (not shown), for example, a flow control valve, a drop monitor system, a syringe pump, or a peristaltic flow control system. The fluid flow modulator is configured to allow controlling the amount of fluid delivered into ablation catheter 600 per unit time, and thereby to effect the fluid's flow rate in extended inlet channel 682. When vacuum source 710 is switched on and suction port 636 is fluidly sealed, the fluid modulator may be used to effect the fluid's propagation rate (flow rate), i.e. via both extended inlet channel 682 and extended outlet channel 684.

Fluid source 720 is further configured to introduce fluid at a controllable introduction temperature, e.g. a coolant at a fixed temperature, into fluid inlet port 692. Accordingly, fluid source 720 may include refrigeration means and a temperature sensor (both not shown).

According to some embodiments, vacuum source 710 and fluid source 720 are interchangeably fluidly coupled to vacuum port 690 in a controllable manner That is to say, when vacuum source 710 is fluidly coupled to vacuum port 690, fluid source 720 is decoupled from vacuum port 690. And when fluid source 720 is fluidly coupled to vacuum port 690, vacuum source 710 is fluidly decoupled from vacuum port 690. In particular, fluid source 720 remains coupled to fluid inlet port 692 even when also coupled to vacuum port 690. In such embodiments, the flow modulator is configured for a slow release of fluid into both inlet port 692 and vacuum port 690, such as to fix the fluid pressure at suction port 636 to slightly above blood pressure, as elaborated on hereinbelow. The interchangeable coupling may be effected, for example, using a valve switch (not shown), which in some embodiments may be actuated hydraulically (e.g. due to fluid pressure), while in other embodiments it may be electrically powered.

In embodiments wherein extended outlet channel 684 includes an additional port (not shown) beyond vacuum port 690, fluid source 720 may be fluidly coupled to the additional port. In such embodiments, both fluid inlet port 692 and the additional port may be used in conjunction to fix and maintain the pressure at suction port 636 at slightly above blood pressure.

Electric power source 730 includes an AC signal generator (not shown). The AC signal generator is electrically coupled via a positive terminal thereof (not shown), to ablation electrode 610, and via a negative port thereof, to a ground electrode (both not shown), such as the ground electrode described in the description of FIG. 1. In some embodiments, the AC signal generator is configured to generate a controllable RF current.

According to some embodiments, the AC signal generator is used to supply power to the sensors located at ablation catheter tip 602 and/or along catheter tubing assembly 604, as detailed above in the description of ablation catheter 600. In some embodiments, electric power source 730 includes additional electric power supply means beyond the AC signal generator, which are used to power some or all of the sensors. In some embodiments, electric power source 730 may power one or more of vacuum source 710, fluid source 720, controller 740, and display 750.

Controller 740 includes a control circuitry and a user interface (both not shown).

Controller 740 is operatively associated with ablation catheter 600, vacuum source 710, fluid source 720, electric power source 730, and optionally with display 750. The control circuitry is configured to receive sensed data from, and in some embodiments to send instructions to, the sensors on catheter tip 602, via the data transmission wires extending along extended tubular member 680. The control circuitry is further configured to send instructions to vacuum source 710, fluid source 720, and electric power source 730. The instructions may include commands input via the user interface, such as to instruct vacuum source 710 to apply suction, to instruct the AC signal generator to generate an RF current to begin ablation, and so on.

In embodiments including the valve switch, the control circuitry may be configured to instruct the valve switch to switch vacuum port 690 fluid coupling, e.g. from fluid source 720 to vacuum source 710. In embodiments including display 750, the control circuitry may be configured to send some or all of the sensed data, either raw or processed, to display 750 to be displayed thereon.

The instructions may also be prompted by sensed data received from the sensors. For example, the control circuitry may be configured to instruct the fluid flow modulator to increase the rate at which the coolant is introduced into fluid inlet port 692 when the temperature sensor readings are above a threshold sensor. More generally, as a function of the received sensed data, the control circuitry may be configured to (a) instruct vacuum source 710 to modify the strength of the suction (e.g. due to readings from the pressure sensor), (b) instruct fluid source 720 to modify the fluid introduction rate and temperature, (c) instruct electric power source 730 to modify the intensity and/or frequency of the generated AC signal (e.g. the RF current induced through ablation electrode 610).

In some embodiments, the control circuitry may include elementary electronic circuits configured to implement some or all of the above-listed functionalities of the control circuitry. In some embodiments, the control circuitry may include application specific integrated circuitry (ASIC). In some embodiments, the control circuitry may include a processor and a non-transitory memory. The processor may include a field-programmable gate array (FPGA), firmware, and/or the like. The user interface may include buttons, knobs, switches, and/or a touch screen. In some embodiments, controller 740 is coupled to an external power source (not shown) and powers the sensors in ablation catheter 600.

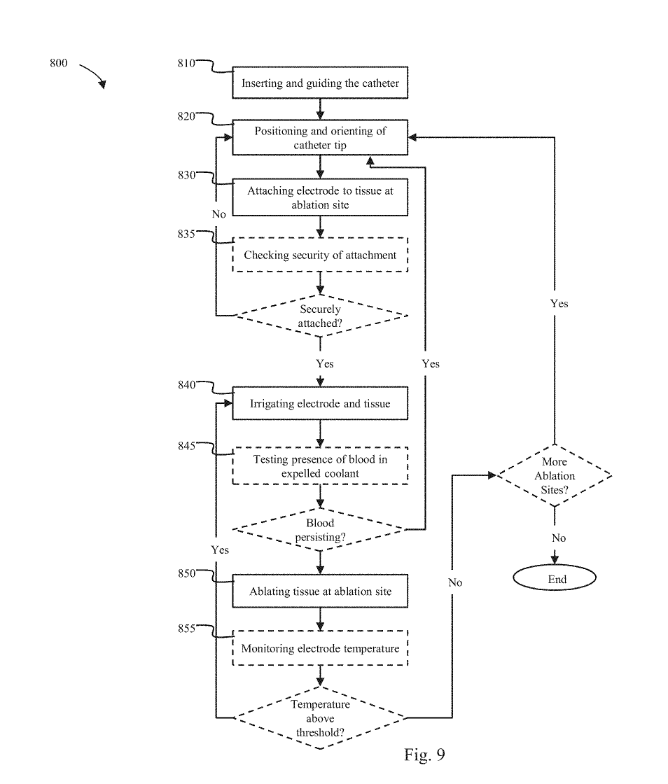

FIG. 9 schematically depicts a flow chart describing an exemplary embodiment of a method 800 for catheter ablation. For concreteness, method 800 is described with respect to endo-cardiac ablation, but the skilled person will understand that method 800 teachings may be applied to other applications involving tissue ablation. For simplicity, method 800 is described with reference to ablation catheter 600 (as shown in FIGS. 7A-7E) and the block diagram depicted in FIG. 8. However, the skilled person will understand that method 800 may be implemented using ablation catheters other than ablation catheter 600, in particular, ablation catheters including a catheter tip, such as catheter tip 100, 200, 300, 400, 500 or similar thereto. Method 800 includes:
A step 810 of inserting extended tubular member 680 into a subject's body and guiding ablation catheter tip 602 into the subject's left atrium proximately to a pulmonary vein opening. Step 810 may be performed using standard techniques known in the art of the cardiac catheters, particularly, endo-cardiac ablation catheters. A ground electrode, electrically coupled to the negative terminal of electric power source 730 AC signal generator, is placed on the subject's body, typically on the subject's back, as explained in the description of FIG. 1.
A step 820 of positioning and orienting ablation catheter tip 602, such that ablation electrode 610 faces a first tissue ablation site, such as tissue ablation site 182, on a target tissue, such as target tissue 180 (shown in FIGS. 2C-2D). The target tissue may be identified, for example, using mapping and sensing electrode rings 654, as explained hereinabove in the description of ablation catheter 600.
A step 830 of securing ablation electrode 610 to the target tissue at the first tissue ablation site. The securing is achieved by applying a vacuum force along extended outlet channel 684 (using vacuum source 710), and thereby generating suction at suction port 636. Due to the suction, ablation electrode 610 is attached to the tissue ablation site. Further, suction port 636 is blocked by tissue adjacent to the first tissue ablation site, such as adjacent tissue 184, and, consequently, a closed irrigation zone, such as closed irrigation zone S1 (shown in FIG. 2C), is formed.
A step 840 of irrigating the closed space with a coolant introduced via fluid inlet port 692 (e.g. the coolant being supplied by fluid source 720). A flow rate of the coolant may be determined based on sensed data, e.g. pressure readings from a pressure sensor mounted on distal member end 614.
A step 850 of ablating the tissue at the first tissue ablation site and forming a lesion thereon. A current (e.g. an RF current) is generated by electric power source 730 AC signal generator. The current runs through ablation electrode 610 and via the tissue at the first tissue ablation site to the ground electrode. Once the tissue has been ablated, step 820 (and subsequent steps) are be repeated with respect to tissue at a second tissue ablation site, e.g. adjacent to the first tissue ablation site, and so on, until all of the target tissue has been ablated.

During steps 810 and 820 and prior to step 830, as well as during the pulling out of extended tubular member 680 once all the target tissue has been ablated, the pressure at suction port 636 is adjusted to and maintained at slightly above blood pressure. In addition to being fluidly coupled to fluid inlet port 692, fluid source 720 is also fluidly coupled to extended outlet channel 684 (e.g. via vacuum port 690 or an additional port fluidly coupled to proximal outlet channel extension end 676). The flow modulator in fluid source 720 is set to slowly release fluid into ports 690 and 692. The fluid release rate is adjusted until the pressure sensor at distal member end 614 signals that the desired pressure has been reached, and then maintained at the desired pressure (and, if need be, readjusted). A typical fluid release rate is about 2 mL per minute. Fixing the blood pressure at suction port 636 to slightly above blood pressure prevents the draining of blood through extended outlet channel 684. When step 830 is about to be applied, fluid source 720 is fluidly decoupled from extended outlet channel 684 (and vacuum source 710 is coupled to extended outlet channel 684).

According to some embodiments, method 800 may further include any of the following steps:
Following step 830, a step 835 of testing whether ablation electrode 610 has been securely attached to the target tissue, using, for example, the force sensor on/in inlet channel cap 640. If not, then step 820 and subsequent steps are repeated.
Following commencement of the irrigation in step 840, a step 845 of testing for blood in the coolant expelled via vacuum port 690. If a presence of blood persists, then step 820, and subsequent steps are repeated. A continued presence of blood in the expelled coolant may indicate that ablation electrode 610 is not properly secured (well attached) to the tissue ablation site and that the closed irrigation zone is in fact not fluidly sealed from the left atrium chamber (or from any other body cavity in applications involving, for example, the ablation of non-cardiac tissue). The expelled coolant may be continuously monitored for signs of blood throughout the duration of the ablation, e.g. also in steps subsequent to step 845, in particular, during step 850. At any sign of blood, the ablation is stopped. A sudden appearance of blood in the expelled coolant may indicate that the attachment of ablation electrode 610 to the tissue ablation site is no longer secure and step 820 and subsequent steps may be repeated. In some embodiments, a sudden appearance of blood may also indicate unintended damage to the target tissue, and consequent bleeding. In some embodiments, the flow rate of the expelled coolant may additionally/alternatively be continuously monitored.
Following commencement of the ablation in step 850, a step 855 of monitoring the temperature of the ablation electrode 610, using the temperature sensor at inlet channel cap 640. If the temperature rises above a threshold temperature the ablation is stopped (i.e. electric power source). Step 840 is then repeated to increase the flow-rate of the coolant.

In some embodiments, simultaneously with the securing of ablation electrode 610 to the target tissue in step 830 (or in step 835), the coolant is introduced into fluid inlet port 692 by fluid source 720, such as to induce a low-rate flow. Fluid expelled via vacuum port 690 is monitored for persistent signs of blood, essentially as described above in step 840. A low-rate flow facilitates a high degree of control of catheter tip 602 in the attaching of ablation electrode 610 to the target tissue, which may be difficult in higher flow-rates, as required for cooling ablation electrode 610 during ablation. If blood persists in the expelled fluid, then step 820 and subsequent steps are repeated. Otherwise, step 840 is initiated and the flow-rate is increased.

It is noted that ablation catheters, such as ablation catheters 10 and 600, may be used to provide continuous monitoring and feedback regarding the security of the attachment of the ablation electrode to a target tissue (e.g. target tissue 180), even when the suction at the suction port(s) is not by itself sufficiently strong to secure the ablation electrode to the target tissue. For simplicity, this option of continuous monitoring is described with reference to ablation catheter 600 and ablation setup 700. However, the skilled person will understand that continuous monitoring may be implemented using ablation catheters other than ablation catheter 600, particularly ablation catheters including a catheter tip, such as catheter tip 100, 200, 300, 400, 500, or similar thereto.

In some embodiments, the securing of ablation electrode 610 to the target tissue may be effected in part, or even primarily, manually by a person guiding the ablation catheter: Once steps 810 and 820 have been performed (i.e. once ablation electrode 610 is facing a tissue ablation site, such as tissue ablation site 182), extended tubular member 680 is distally pushed, such as to cause ablation catheter tip 602 to press against the target tissue, and in particular, to cause ablation electrode 610 to press against the tissue at the tissue ablation site. Ablation catheter tip 602 is maintained pressed against the tissue for the duration of the ablation at the tissue ablation site. In some embodiments, the securing may be effected in part, or even primarily, automatically by a robotic guiding system.

In conjunction with the manual or automatic manipulation of the ablation, catheter tip suction is applied at suction port 636. While the pressing of ablation catheter tip 602 against the target tissue may by itself result in the formation of a closed irrigation zone, such as closed irrigation zone S1, the suction applied at suction port 636 may help ensure that the closed irrigation zone is, and remains, fluidly sealed.

A continued presence of blood in the expelled coolant may indicate that ablation electrode 610 has not been securely attached to the target tissue. A sudden presence of blood in the expelled coolant, after a continuous period wherein the expelled coolant was blood-free, may indicate that ablation electrode 610 is no longer securely attached to the target tissue. For example, when the securing is at least in part effected manually, the sudden appearance of blood may indicate that the person guiding the catheter has eased the distal pressing of extended tubular member 680. Such an easing of the distal pressing may lead to ablation electrode 610 becoming detached from the tissue ablation site and to the fluidic unsealing of the closed irrigation zone. In some embodiments, the sudden appearance of blood may indicate unintended damage to the target tissue.

The skilled person will understand that the continuous monitoring of an expelled irrigant, as described hereinabove, is not limited to applications involving tissue ablation, and may be used in other applications wherein an operative element or medical probe needs to be secured to a target tissue in a body cavity.

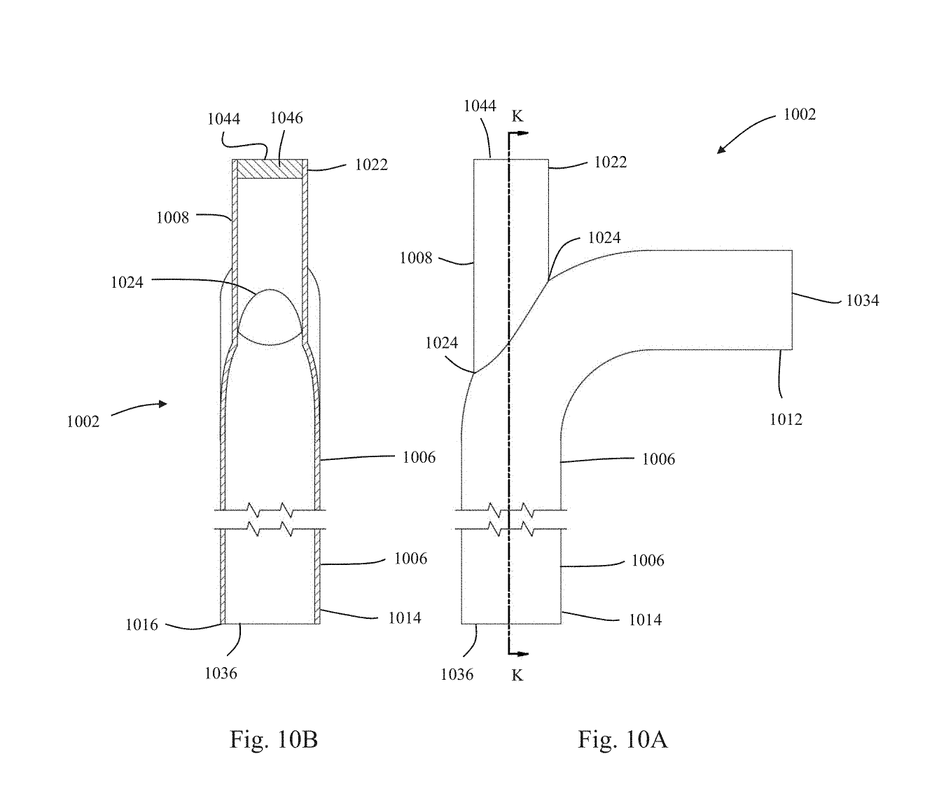

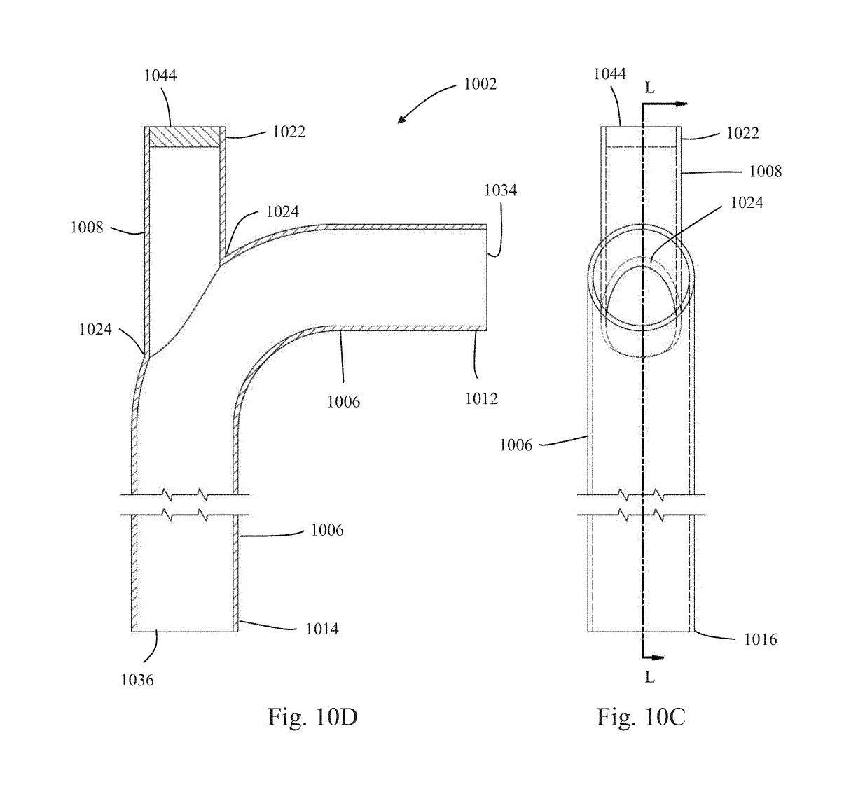

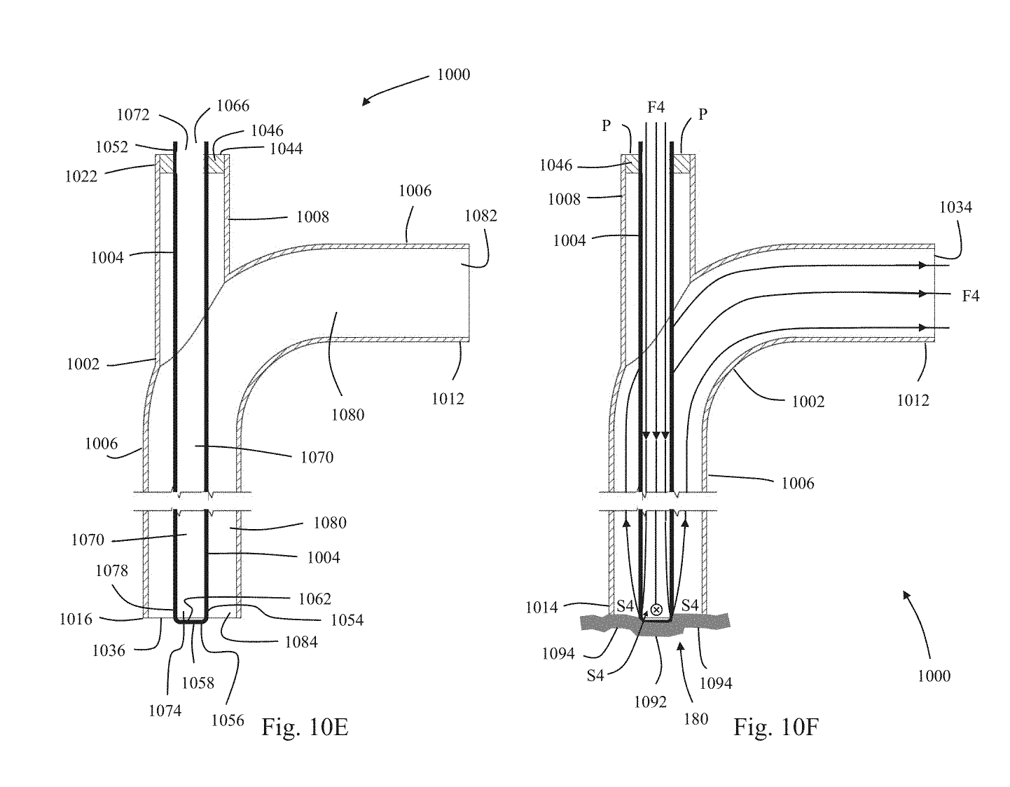

FIGS. 10A-10F schematically depict an exemplary embodiment of a catheter ablation assembly 1000 (shown in full in FIGS. 10E-10F). FIG. 10A provides a side-view of a delivery catheter 1002. FIG. 10B provides a cross-sectional front-view of delivery catheter 1002 taken along a line K-K (indicated in FIG. 10A). FIG. 10C provides a back-view of delivery catheter 1002. FIG. 10D provides a cross-sectional side-view of delivery catheter 1002 taken along a line L-L (indicated in FIG. 10C). FIGS. 10E-10F provide a cross-sectional side-view of delivery catheter 1002, taken along line L-L, with an ablation catheter tube 1004 inserted into delivery catheter 1002.

As shown in FIG. 10A, delivery catheter 1002 includes an elongate member 1006, which is tubular, and a catheter insertion tube 1008. Elongate member 1006 extends from a proximal member end 1012 to a distal member end 1014. A distal member extremity 1016 consists of the distal edge of distal member end 1014. Catheter insertion tube 1008 extends from a proximal insertion tube end 1022 to a tubing junction 1024. Catheter insertion tube 1008 is joined to, and fluidly connects to, elongate member 1006 at tubing junction 1024.

A vacuum port 1034 is mounted on proximal member end 1012, and a suction port 1036 is mounted on distal member end 1014. Vacuum port 1034 is configured to be fluidly coupled to a vacuum source, such as vacuum source 710, and thereby to induce suction at suction port 1036.

A catheter insertion port 1044 is mounted on proximal insertion tube end 1022. Proximal insertion tube end 1022 includes a sealing membrane 1046 (shown in FIGS. 10B-10F). Sealing membrane 1046 covers catheter insertion port 1044 and thereby fluidly seals proximal insertion tube end 1022. Sealing membrane 1046 is configured to allow perforation thereof and insertion therethrough of a narrow member, such as ablation catheter tube 1004 (depicted in FIGS. 10E-10F). Sealing membrane 1046 is further configured to envelop the inserted narrow member, such as to maintain fluidic sealing around the narrow member, while simultaneously allowing the narrow member to be further inserted (e.g. distally pushed). FIGS. 10E-10F depict catheter ablation assembly 1000. Catheter ablation assembly includes delivery catheter 1002 and ablation catheter tube 1004. FIGS. 10E-10F depict ablation catheter tube 1004 inserted into catheter insertion tube 1008, with sealing membrane 1046 fluidly sealing an area P, which consists of the area surrounding ablation catheter tube 1004 at proximal insertion tube end 1022.

According to some embodiments, tubing junction 1024 may be located inside a catheter handle, such as catheter handle 20, with vacuum port 1034 and catheter insertion port 1044 providing exemplary embodiments of vacuum port 54 and one of additional ports 82, respectively.

FIG. 10E schematically depicts delivery catheter 1002 with ablation catheter tube 1004 inserted into elongate member 1006 via catheter insertion tube 1008. Ablation catheter tube 1004 is an open loop cooling ablation catheter tube. Ablation catheter tube 1004 extends from a proximal ablation tube end 1052 to a distal ablation tube end 1054. A distal ablation tube extremity 1056 consists of the distal edge of distal ablation tube end 1054. An ablation electrode 1058 is positioned on/in distal ablation tube end 1054, such as to be at least partially exposed on distal ablation tube extremity 1056. Ablation electrode 1058 is thermally coupled to an internal surface 1062 of distal ablation tube end 1054, essentially as described above in the description of ablation catheter tip 100. (Internal surface 1062 is parallel to distal ablation tube extremity 1056.) In some embodiments, ablation electrode 1058 is at least partially exposed on internal surface 1062. A fluid inlet port 1066 is mounted on proximal ablation tube end 1052. Fluid inlet port 1066 is configured to be fluidly coupled to a fluid source, such as fluid source 720, and thereby to deliver fluid (e.g. a coolant) at distal ablation tube end 1054.

Ablation catheter tube 1004 defines therein an inlet channel 1070, longitudinally extending from a proximal inlet channel end 1072, located at proximal ablation tube end 1052, to a distal inlet channel end 1074, located at distal ablation tube end 1054. Distal inlet channel end 1074 is fluidly connected to fluid inlet port 1066 via inlet channel 1070.

Distal ablation tube end 1054 includes fluid openings (not shown), fluidly connecting distal inlet channel end 1074 to the outside of distal ablation tube end 1054. The fluid openings are configured to at least partially discharge an irrigant, arriving at distal inlet channel end 1074 (via inlet channel 1070), to the outside of distal ablation tube end 1054, and thereby to wash ablation electrode 1058 also from the outside of distal ablation tube end 1054 (that is to say, not only on internal surface 1062).

When the irrigant is a coolant and a current is induced through ablation electrode 1058, the washing on the outside of distal ablation tube end 1054 helps in cooling ablation electrode 1058. In some embodiments, the fluid openings may extend through ablation electrode 1058, thereby helping to further cool ablation electrode 1058 when a current is passed therethrough. For example, the fluid openings may extend between internal surface 1062 and a circumferential surface 1078 of distal ablation tube end 1054, via ablation electrode 1058. When ablation electrode 1058 is secured to a target tissue, such as target tissue 180, the coolant discharged through the fluid openings may wash and cool some of the target tissue.

When ablation catheter tube 1004 is fully inserted into elongate member 1006 (that is to say, when distal ablation tube extremity 1056 is located at distal member extremity 1016, or distally extends slightly farther than distal member extremity 1016, e.g. by about 1 mm to about 5 mm), elongate member 1006 and ablation catheter tube 1004 define an outlet channel 1080. Outlet channel 1080 includes the space inside elongate member 1006, which is outside ablation catheter tube 1004. Outlet channel 1080 extends from a proximal outlet channel end 1082, located at proximal member end 1012, to a distal outlet channel end 1084, located at distal member end 1014. Proximal outlet channel end 1082 is fluidly connected to vacuum port 1034. Distal outlet channel end 1084 is fluidly connected to suction port 1036.

A temperature sensor (not shown) is also positioned on/in distal ablation tube end 1054. Additional sensors, as listed in the description of ablation catheter tip 100, may also be positioned on/in distal ablation tube end 1054 and/or distal member end 1014. Electrical wires (not shown) extend through ablation catheter tube 1004 and supply power to ablation electrode 1058, and in some embodiments to the temperature sensor. In some embodiments, the electrical wires, or additional electrical wires extending, for example, through outlet channel 1080 or the walls of elongate member 1006, supply power to some or all of the sensors. Data transmission wires (not shown), extending through ablation catheter tube 1004, outlet channel 1080, and/or elongate member 1006 walls, transmit sensed readings, e.g. temperature readings by the temperature sensor, to an external control circuitry, as described in the description of FIG. 8. Optionally, the data transmission wires may further transmit instructions from the external control circuitry to some or all of the sensors.

FIG. 10F schematically depicts catheter ablation assembly 1000 in operation in the treatment of AF, according to some embodiments: Distal member end 1014 is guided into the left atrium chamber proximately to a pulmonary vein opening. According to some embodiments, wherein distal member end 1014 includes mapping and sensing electrodes (similarly to ablation catheter tip 100), the mapping and sensing electrodes may be used to identify target tissue 180, and determine a location thereof on the pulmonary vein opening. The position and orientation of distal member end 1014 is adjusted such as to face a tissue ablation site 1092 on target tissue 180.

Ablation catheter tube 1004 is inserted into catheter insertion tube 1008, via sealing membrane 1046. Ablation catheter tube 1004 is guided through elongate member 1006 until distal ablation tube extremity 1056 reaches, or distally slightly extends beyond, distal member extremity 1016.

When suction is applied via suction port 1036, ablation electrode 1058 is secured to tissue ablation site 1092. Suction port 1036 and an adjacent tissue 1094, surrounding tissue ablation site 1092, are drawn towards each other. Suction port 1036 is thereby covered by adjacent tissue 1094 and thereby blocked, and a closed (or effectively closed) irrigation zone S4 is formed (shown in FIG. 10F). Closed irrigation zone S4 includes the space about distal member end 1014, which is fluidly disconnected from the left atrium chamber by target tissue 180 at tissue ablation site 1092 and particularly by adjacent tissue 1094.

Once ablation electrode 1058 has been secured to target tissue 180 and closed, irrigation zone S4 has been sealed, a coolant (or in some embodiments, some other type of irrigant) is introduced into inlet channel 1070 (i.e. into ablation catheter tube 1004) via fluid inlet port 1066. The coolant flows until distal inlet channel end 1074, wherefrom it is directed via the fluid openings in distal ablation tube end 1054 into distal outlet channel end 1084. Due to the vacuum force acting proximally along outlet channel 1080, the resultant blocking of suction port 1036, and the fluidic sealing provided by sealing membrane 1046 around catheter ablation tube 1004 at proximal insertion tube end 1022, substantially all of the coolant is made to proximally flow along outlet channel 1080, exiting via vacuum port 1034. Arrows F4 represent the coolant's flow direction. The coolant washes away blood in closed irrigation zone S4. In particular, the coolant will wash away ablation byproducts, such as char.

Following the introduction of the coolant, the ablation is started by applying a current, e.g. an RF current, through ablation electrode 1058. Consequently, ablation electrode 1058 and target tissue 180, particularly at tissue ablation site 1092, as well as adjacent tissue 1094, begin heating. The coolant is washed against internal surface 1062, thereby cooling ablation electrode 1058, that is to say, absorbing heat from ablation electrode 1058. In addition, some of the coolant passing through the outlet openings cools ablation electrode 1058 from the outside of distal ablation tube end 1054 (e.g. on distal ablation tube extremity 1056), as described hereinabove. Further, some of the coolant passing through the fluid openings may be washed and/or sprayed against target tissue 180 and/or adjacent tissue 1094, thereby cooling adjacent tissue 1094.

According to an aspect of some embodiments, there is provided an ablation catheter tip (for example, 14, 100, 200, 300, 400, 500, 1500, 602). The ablation catheter tip includes:
A tip body (for example, 101, 402, 502, 606), having a proximal tip body end (for example, 112, 412, 512, 612) and a distal tip body end (for example, 114, 414, 514, 1514, 614).
An inlet channel (for example, 104, 440, 504a, 504b, 608), having a proximal inlet channel end (for example, 122, 442, 522a, 522b, 622) and a distal inlet channel end (for example, 124, 444, 524a, 524b, 624), the inlet channel being longitudinally disposed within the tip body.
An outlet channel (for example, 130, 430, 508a, 508b, 630), having a proximal outlet channel end (for example, 132, 432, 532a, 532b, 632) and a distal outlet channel end (for example, 134, 434, 534a, 534b, 634), the outlet channel being longitudinally disposed within the tip body.
A suction port (for example, 136, 452, 538a, 538b, 636), located at the distal tip body end and fluidly coupled to the distal outlet channel end.
An ablation electrode (for example, 16, 106, 406, 506, 610) positioned at the distal tip body end.

The suction port is configured to secure a target tissue (for example, 180), at a tissue ablation site (for example, 182, 482, 582) on the target tissue, to the ablation electrode by applying a vacuum force via the outlet channel when the distal tip body end is proximate to or in contact with the target tissue.

The inlet channel and the outlet channel are fluidly coupled at the distal tip body end such that the fluid coupling is maintained when the suction port is covered, thereby facilitating propagating a fluid from the inlet channel to the outlet channel and expelling the fluid via the proximal outlet channel end, when the vacuum force secures (i) the ablation electrode to the tissue ablation site and (ii) an adjacent tissue (for example, 184, 484, 584), to the tissue ablation site, to the suction port.

According to some embodiments, the distal tip body end is configured to induce direct and/or indirect (indirect coupling, for example, may be provided via cap portion 152) thermal coupling between the ablation electrode and a fluid present at the distal inlet channel end, at the distal outlet channel end, and/or in between the channels at the distal tip body end, and thereby to controllably effect a temperature of the ablation electrode by propagating the fluid at a controllable introduction temperature via the inlet channel and the outlet channel, through the distal tip body end.

According to some embodiments, the inlet channel and the outlet channel are fluidly connected via an opening (for example, 148, 248, 454, 648), a duct, or a recess (for example, 552a-552d, 1552a-1552d).

According to some embodiments, the ablation catheter tip may be used for treating AF.

According to an aspect of some embodiments, there is provided a catheter ablation method (800). The catheter ablation method includes the steps of:
Inserting (810) into a subject's body a catheter (for example, 10, 600) including
   a catheter tip (for example, 14, 100, 200, 300, 400, 500, 1500, 602) with an ablation electrode (for example, 16, 106, 406, 506, 610) positioned on/in a distal end thereof (for example, 114, 414, 514, 1514, 614);
   an inlet channel (for example, 682) and an outlet channel (for example, 684) both extending along the catheter until the catheter tip distal end and fluidly coupled at the catheter tip distal end; and
   a suction port (for example, 136, 452, 538a, 538b, 636) mounted on the catheter tip distal end and fluidly coupled to the outlet channel.

The catheter tip being configured such that the fluid coupling of the inlet channel and outlet channel at the catheter tip distal end is maintained when the suction port is covered.
Positioning and orienting (820) the catheter tip, such that the ablation electrode faces a tissue ablation site (for example, 182, 482, 582) on a target tissue (180) in a body cavity (for example, 62);
Securing (830) the ablation electrode to the tissue ablation site by applying a vacuum force along the outlet channel, thereby covering the suction port with a tissue adjacent (for example, 184, 484, 584) to the tissue ablation site and fluidly sealing the catheter tip from the body cavity.
Propagating (840) an irrigant through the inlet channel and the outlet channel, via the catheter tip distal end, wherein the irrigant washes against the adjacent tissue covering the suction port.
Ablating (850) the tissue at the tissue ablation site.

According to some embodiments, the ablation catheter method may be used for treating AF.

### Experimental Example

The ablation catheter and methods described herein above, were experimentally tested for performing tissue ablation in a target site of a myocardial tissue, while avoiding excessive heating of the tissue which may lead to intramyocardial explosion which is indicated by a steam-pop. As used herein the term “steam pop” refers to the audible sound produced by intramyocardial explosion when tissue temperature reaches 100 degree Celsius (° C.), leading to the production of gas. It is a potentially severe complication of radiofrequency ablation because it has been associated with cardiac perforation and ventricular septal defect.

Specifically, tissue ablation in a target site of a pig's heart tissue was performed while securing the ablation electrode to the tissue ablation site by applying a vacuum force (test group) or without vacuum (control). To this end, a tissue of a pig's heart was placed in a vessel filled with warm water. An ablation catheter was introduced through a delivery catheter which was coupled to a vacuum source. In the test group experiments, the delivery catheter was secured to the tissue ablation site by applying a vacuum force. Next, a tip of an ablation catheter was positioned such that the ablation electrode faced the tissue ablation site. Further, irrigation with saline was performed in a flow rate of 30 cubic centimeter per minute (cm³/min). Ablation was performed by applying a power of 2, 5, 10, 15, 20, or 30 Watts for a time duration of up to 120 seconds until tissue ablation was reached or alternatively until a steam-pop occurred.

As demonstrated in table 1, securing the ablation electrode to the tissue ablation site by applying a vacuum force advantageously facilitates reaching tissue ablation and avoiding steam-pop. In addition, results of initial validation experiments suggested that addition of a mechanism configured to allow maneuvering/movement of the ablation catheter relative to the delivery insertion catheter may improve ability to couple the ablation catheter to the tissue and may further improve tissue ablation effectiveness.

TABLE 1
Experimental results

| Securing the | | | |
|---|---|---|---|
| ablation electrode | | | |
| to the tissue | Applied | | |
| ablation site by | Power | Tissue ablation or | |
| applying vacuum | (Watt) | steam-pop | Remarks |
| Yes | 2 | No tissue ablation or | High |
| | | steam-pop following | resistance |
| | | 120 seconds of operation | |
| | 5 | Tissue ablation | Resistance |
| | | following 70 seconds | dropped |
| | | of operation | during |
| | | | ablation |
| | 10 | Tissue ablation following | Superficial |
| | | less than 30 seconds | scar |
| | | of operation | |
| | 15 | Tissue ablation following | Superficial |
| | | less than 30 seconds | scar |
| | | of operation | |
| | 20 | Tissue ablation following | Superficial |
| | | less than 30 seconds | scar |
| | | of operation | |
| | 30 | Tissue ablation following | Superficial |
| | | less than 30 seconds | scar |
| | | of operation | |
| No | 2 | No tissue ablation or | Superficial |
| | | steam-pop following | scar |
| | | 120 seconds of operation | |
| | 5 | No tissue ablation or | Superficial |
| | | steam-pop following | scar |
| | | 120 seconds of operation | |
| | 10 | Steam-pop occurred | Deep scar |
| | | following 30 seconds | |
| | | of operation | |
| | 15 | Steam-pop occurred | Deep scar |
| | | following 30 seconds | |
| | | of operation | |
| | 20 | Steam-pop occurred | Deep scar |
| | | following 30 seconds | |
| | | of operation | |
| | 30 | Steam-pop occurred | Deep scar |
| | | following 30 seconds | |
| | | of operation | |

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What we claim is:

1. An ablation catheter tip comprising
a tip body, having a proximal tip body end and a distal tip body end; an inlet channel, having a proximal inlet channel end and a distal inlet channel end, said inlet channel being longitudinally disposed within said tip body;
an outlet channel, having a proximal outlet channel end and a distal outlet channel said outlet channel being longitudinally disposed within said tip body;
a suction port, located at said distal tip body end and fluidly coupled to said distal outlet channel end; and
an ablation electrode positioned at said distal tip body end; wherein said suction port is configured to secure a target tissue, at a tissue ablation site on the target tissue, to said ablation electrode by applying a vacuum force via said outlet channel when said distal tip body end is proximate to or in contact with the target tissue; and
wherein said inlet channel and said outlet channel are fluidly coupled at said distal tip body end such that the fluid coupling is maintained when said suction port is covered, thereby facilitating propagating a fluid from said inlet channel to said outlet channel and expelling the fluid via said proximal outlet channel end, when the vacuum force secures (i) said ablation electrode to the tissue ablation site and (ii) tissue, adjacent to the tissue ablation site, to said suction port.

2. The ablation catheter tip of claim 1 wherein said distal tip body end is configured to induce direct and/or indirect thermal coupling between said ablation electrode and a coolant fluid present at said distal inlet channel end, at said distal outlet channel end, and/or in between said channels at said distal tip body end, and thereby to controllably effect a temperature of said ablation electrode by propagating the coolant fluid at a controllable introduction temperature via said inlet channel and said outlet channel, through said distal tip body end.

3. The ablation catheter tip of claim 1, wherein said inlet channel and said outlet channel are fluidly connected via an opening, duct, or recess.

4. (canceled)

5. The ablation catheter tip of claim 1, wherein said inlet channel extends between said proximal tip body end and said distal tip body end and wherein said outlet channel extends between said proximal tip body end and said distal tip body end.

6. (canceled)

7. The ablation catheter tip of claim 1, wherein said suction port at least partially circumscribes said ablation electrode.

8. The ablation catheter tip of claim 7 wherein said tip body and said inlet channel are tubular, and said outlet channel is defined by said tip body and inlet channel, and comprises a space between said tip body and said inlet channel.

9. The ablation catheter tip of claim 8 wherein said inlet channel further comprises an inlet channel cap, mounted on said distal inlet channel end; and at least one fluid opening, located at said distal inlet channel end, wherein said ablation electrode is positioned in/on said inlet channel cap, such as to be at least partially exposed, and wherein said fluid opening fluidly connects said inlet channel to said outlet channel.

10. The ablation catheter tip of claim 9 wherein said at least one fluid opening comprises two or more fluid openings, being annularly disposed about said inlet channel.

11. The ablation catheter tip of claim 7 further comprising an inlet tube longitudinally disposed within said tip body, extending from a proximal inlet tube end to a distal inlet tube end; and an inner core longitudinally disposed within said inlet tube;
wherein said outlet channel is defined by said tip body and said inlet tube, and comprises a first space between said tip body and said inlet tube;
wherein said inlet channel is defined by said inlet tube and said inner core, and comprises a second space between said inlet tube and said inner core;
wherein said tip body extends distally farther than said inlet tube;
wherein said inner core extends distally at least as much as said tip body; and wherein said ablation electrode is positioned on/in a core tip of said inner core.

12. The ablation catheter tip of claim 6 further comprising:
a second inlet channel, having a proximal second inlet channel end and a distal second inlet channel end, said second inlet channel being longitudinally disposed within said tip body;
a second outlet channel, having a proximal second outlet channel end and a distal second outlet channel end, said second outlet channel being longitudinally disposed within said tip body; and
a second suction port, located at said distal tip body end and fluidly coupled to said distal second outlet channel end;
wherein said distal tip body end comprises four recesses, each of said recesses extending from a respective proximal inlet channel end to a respective distal outlet channel end, such as to circumscribe said ablation electrode, said recesses being configured to maintain fluid connectivity between said inlet channels and said outlet channels when said recesses, said inlet channel ends, and said suction ports are covered at a distal tip body extremity of said distal tip body.

13. (canceled)

14. (canceled)

15. The ablation catheter tip of claim 1, for use in treatment of atrial fibrillation.

16. The ablation catheter tip of claim 1, wherein said ablation electrode is radially, axially, longitudinally moveable relative to said distal tip body.

17. (canceled)

18. An ablation catheter comprising:
an elongate member, being flexible, having a proximal member end and a distal member end;
an inlet channel, having a proximal inlet channel end and a distal inlet channel end, at least a distal portion of said inlet channel being longitudinally disposed within said elongate member;
an outlet channel, having a proximal outlet channel end and a distal outlet channel end, said outlet channel being longitudinally disposed within said elongate member;
a suction port mounted on said distal outlet channel end;
a vacuum port, mounted on said proximal outlet channel end; a fluid inlet port mounted on said proximal inlet channel end; and an ablation electrode positioned at said distal member end; wherein said fluid inlet port is configured to be fluidly coupled to a fluid source;
wherein said vacuum port is configured to be fluidly coupled to a vacuum source;
wherein said suction port is configured to secure a target tissue, at a tissue ablation site on the target tissue, to said ablation electrode by applying a vacuum force via said outlet channel when said distal member end is proximate to or in contact with the target tissue; and
wherein said inlet channel and said outlet channel are fluidly coupled at said distal member end such that the fluid coupling is maintained when said suction port is covered, thereby facilitating propagating a fluid from said inlet channel to said outlet channel and expelling the fluid via said vacuum port, when the vacuum force secures (i) said ablation electrode to the tissue ablation site and (ii) a tissue, adjacent to the tissue ablation site, to said suction port.

19. The ablation catheter of claim 18, wherein said distal member end is configured to induce direct and/or indirect thermal coupling between said ablation electrode and a fluid present at said distal inlet channel end, at said distal outlet channel end, and/or in between said channels at said distal member end, and thereby to controllably effect a temperature of said ablation electrode by propagating the fluid at a controllable introduction temperature via said inlet channel and said outlet channel, through said distal member end.

20. (canceled)

21. A catheter ablation method comprising:
inserting into a subject's body a catheter comprising
a catheter tip with an ablation electrode positioned on/in a distal end thereof;
an inlet channel and an outlet channel both extending along the catheter until the catheter tip distal end and fluidly coupled at the catheter tip distal end; and
a suction port mounted on the catheter tip distal end and fluidly coupled to the outlet channel;
wherein the fluid coupling of the inlet channel and outlet channel at the catheter tip distal end is maintained when the suction port is covered;
positioning and orienting the catheter tip, such that the ablation electrode faces a tissue ablation site on a target tissue in a body cavity;
securing the ablation electrode to the tissue ablation site by applying a vacuum force along the outlet channel, thereby covering the suction port with a tissue, adjacent to the tissue ablation site, and fluidly sealing the catheter tip from the body cavity;
propagating an irrigant through the inlet channel and the outlet channel, via the catheter tip distal end, wherein the irrigant washes against the adjacent tissue covering the suction port; and
ablating the tissue at the tissue ablation site.

22. The catheter ablation method of claim 21, wherein the irrigant is a coolant and wherein the catheter tip distal end is configured such that heat generated by the ablation electrode is transferred to the coolant when the coolant flows through the catheter tip distal end, thereby effecting a temperature of said ablation electrode in said step of propagating the irrigant.

23. (canceled)

24. The ablation catheter method of claim 23, further comprising, prior to said step of ablating, testing for a presence of blood in the coolant expelled via the inlet channel, if the presence of blood persists
significantly decreasing the flow of the coolant;
switching off the vacuum force; and
repeating said step of positioning and orienting and subsequent steps.

25. The ablation catheter method of claim 24, further comprising, following said step of ablating, monitoring a temperature of said ablation electrode, if the temperature exceeds a threshold temperature
switching off the current;
increasing the flow of the coolant; and
switching on the current again.

26. The ablation catheter method of claim 21, further comprising, following said step of ablating, if there remain tissue ablation sites that have not been ablated, repeating said step of positioning and orienting the catheter tip and subsequent steps with respect to another tissue ablation site.

27. The ablation catheter method of claim 21, for use in treatment of atrial fibrillation.
